SEQUENCE LISTING (111 sequences)

SEQ ID NO: 1 - protein, 5 aa, Staphylococcus aureus
  MISC_FEATURE (3)..(3): X in position 3 can be any amino acid
  MISC_FEATURE (3)..(3): nucleophilic recognition sequence, X in position 3 can be any amino acid
LPXTG

SEQ ID NO: 2 - protein, 5 aa, Staphylococcus aureus
LPETG

SEQ ID NO: 3 - protein, 5 aa, Staphylococcus aureus
LPATG

SEQ ID NO: 4 - protein, 5 aa, Staphylococcus aureus
  MISC_FEATURE (3)..(3): X in position 3 can be Q or K
  MISC_FEATURE (3)..(3): nucleophilic recognition sequence, X in position 3 can be Q or K
NPXTN

SEQ ID NO: 5 - protein, 5 aa, Staphylococcus aureus
  MISC_FEATURE (1)..(1): X in position 1 can be I or a L
  MISC_FEATURE (1)..(1): nucleophilic recognition sequence, X in position 1 can be I or a L
  MISC_FEATURE (2)..(2): X in position 2 can be a P or an A
  MISC_FEATURE (3)..(3): X in position 3 can be any amino acid
XXXTG

SEQ ID NO: 6 - protein, 5 aa, Staphylococcus aureus
QVPTG

SEQ ID NO: 7 - protein, 5 aa, Staphylococcus aureus
LPNTA

SEQ ID NO: 8 - protein, 5 aa, Staphylococcus aureus
  MISC_FEATURE (3)..(3): X in position 3 can be any amino acid
  MISC_FEATURE (3)..(3): nucleophilic recognition sequence, X in position 3 can be any amino acid
LAXTG

SEQ ID NO: 9 - protein, 5 aa, Staphylococcus aureus
  MISC_FEATURE (1)..(1): X in position 1 can be M, L or V
  MISC_FEATURE (2)..(2): X in position 2 can be P, T, A or S
  MISC_FEATURE (4)..(4): X in position 4 can be A, L, T, S, V or I
XXAXG

SEQ ID NO: 10 - protein, 5 aa, Staphylococcus aureus
  MISC_FEATURE (3)..(3): X in position 3 can be any amino acid
  MISC_FEATURE (3)..(3): nucleophilic recognition sequence, X in position 3 can be any amino acid
LPXTA

SEQ ID NO: 11 - protein, 5 aa, Staphylococcus aureus
NPQTN

SEQ ID NO: 12 - protein, 5 aa, Staphylococcus aureus
  MISC_FEATURE (3)..(3): X in position 3 can be any amino acid
  MISC_FEATURE (3)..(3): nucleophilic recognition sequence, X in position 3 can be any amino acid
LPXSG

SEQ ID NO: 13 - protein, 4 aa, Staphylococcus aureus
GGGG

SEQ ID NO: 14 - protein, 5 aa, Staphylococcus aureus
GGGGG

SEQ ID NO: 15 - protein, 4 aa, Staphylococcus aureus
AAAA

SEQ ID NO: 16 - protein, 5 aa, Staphylococcus aureus
AAAAA

SEQ ID NO: 17 - protein, 5 aa, Artificial Sequence
  beta barrel structure
WTWTW

SEQ ID NO: 18 - protein, 5 aa, Artificial Sequence
  intersequence between block 1 and block 2
  MISC_FEATURE (1)..(1): X in position 1 can be I or L
  MISC_FEATURE (2)..(2): X in position 2 can be P or A
  MISC_FEATURE (3)..(3): X in position 3 can be any amino acid
XXXTG

SEQ ID NO: 19 - protein, 6 aa, Artificial Sequence
  intersequence between block 1 and block 2
  MISC_FEATURE (1)..(1): X in position 1 can be I or L
  MISC_FEATURE (2)..(2): X in position 2 can be P or A
  MISC_FEATURE (3)..(3): X in position 3 can be any amino acid
XXXTGG

SEQ ID NO: 20 - protein, 7 aa, Artificial Sequence
  intersequence between block 1 and block 2
  MISC_FEATURE (1)..(1): X in position 1 can be I or L
  MISC_FEATURE (2)..(2): X in position 2 can be P or A
  MISC_FEATURE (3)..(3): X in position 3 can be any amino acid
XXXTGGG

SEQ ID NO: 21 - protein, 8 aa, Artificial Sequence
  intersequence between block 1 and block 2
  MISC_FEATURE (1)..(1): X in position 1 can be I or L
  MISC_FEATURE (2)..(2): X in position 2 can be P or A
  MISC_FEATURE (3)..(3): X in position 3 can be any amino acid
XXXTGGGG

SEQ ID NO: 22 - protein, 9 aa, Artificial Sequence
  inter sequence between block 1 and block 2
  MISC_FEATURE (1)..(1): X in position 1 can be I or L
  MISC_FEATURE (2)..(2): X in position 2 can be P or A
  MISC_FEATURE (3)..(3): X in position 3 can be any amino acid
XXXTGGGGG

SEQ ID NO: 23 - protein, 5 aa, Artificial Sequence
  inter-sequence between block 1 and block 2
  MISC_FEATURE (1)..(1): X in position 1 can be I or L
  MISC_FEATURE (2)..(2): X in position 2 can be P or A
  MISC_FEATURE (3)..(3): X in position 3 can be any amino acid
XXXTA

SEQ ID NO: 24 - protein, 6 aa, Artificial Sequence
  inter-sequence between block 1 and block 2
  MISC_FEATURE (1)..(1): X in position 1 can be I or L
  MISC_FEATURE (2)..(2): X in position 2 can be P or A
  MISC_FEATURE (3)..(3): X in position 3 can be any amino acid
XXXTAA

SEQ ID NO: 25 - protein, 7 aa, Artificial Sequence
  inter-sequence between block 1 and block 2
  MISC_FEATURE (1)..(1): X in position 1 can be I or L
  MISC_FEATURE (2)..(2): X in position 2 can be P or A
  MISC_FEATURE (3)..(3): X in position 3 can be any amino acid
XXXTAAA

SEQ ID NO: 26 - protein, 8 aa, Artificial Sequence
  inter-sequence between block 1 and block 2
  MISC_FEATURE (1)..(1): X in position 1 can be I or L
  MISC_FEATURE (2)..(2): X in position 2 can be P or A
  MISC_FEATURE (3)..(3): X in position 3 can be any amino acid
XXXTAAAA

SEQ ID NO: 27 - protein, 9 aa, Artificial Sequence
  inter-sequence between block 1 and block 2
  MISC_FEATURE (1)..(1): X in position 1 can be I or L
  MISC_FEATURE (2)..(2): X in position 2 can be P or A
  MISC_FEATURE (3)..(3): X in position 3 can be any amino acid
XXXTAAAAA

SEQ ID NO: 28 - protein, 5 aa, Artificial Sequence
  inter-sequence between block 1 and block 2
  MISC_FEATURE (3)..(3): X in position 3 can be Q or K
NPXTG

SEQ ID NO: 29 - protein, 6 aa, Artificial Sequence
  inter-sequence beween block 1 and block 2
  MISC_FEATURE (3)..(3): X in position 3 can be Q or K
NPXTGG

SEQ ID NO: 30 - protein, 7 aa, Artificial Sequence
  inter-sequence between block 1 and block 2
  MISC_FEATURE (3)..(3): X in position 3 can be Q or K
NPXTGGG

SEQ ID NO: 31 - protein, 8 aa, Artificial Sequence
  inter-sequence between block 1 and block 2
  MISC_FEATURE (3)..(3): X in postion 3 can be Q or K
NPXTGGGG

SEQ ID NO: 32 - protein, 9 aa, Artificial Sequence
  inter-sequence between block 1 and block 2
  MISC_FEATURE (3)..(3): X in position 3 can be Q or K
NPXTGGGGG

SEQ ID NO: 33 - protein, 5 aa, Artificial Sequence
  inter-sequence between block 1 and block 2
  MISC_FEATURE (3)..(3): X in position 3 can be Q or K
NPXTA

SEQ ID NO: 34 - protein, 6 aa, Artificial Sequence
  inter-sequence between block 1 and block 2
  MISC_FEATURE (3)..(3): X in position 3 can be Q or K
NPXTAA

SEQ ID NO: 35 - protein, 7 aa, Artificial Sequence
  inter-sequence between block 1 and block 2
  MISC_FEATURE (3)..(3): X in position 3 can be Q or K
NPXTAAA

SEQ ID NO: 36 - protein, 8 aa, Artificial Sequence
  inter-sequence between block 1 and block 2
  MISC_FEATURE (3)..(3): X in position 3 can be Q or K
NPXTAAAA

SEQ ID NO: 37 - protein, 9 aa, Artificial Sequence
  inter-sequence between block 1 and block 2
  MISC_FEATURE (3)..(3): X in position 3 can be Q or K
NPXTAAAAA

SEQ ID NO: 38 - protein, 5 aa, Artificial Sequence
  inter-sequence between block 1 and block 2
QVPTG

SEQ ID NO: 39 - protein, 6 aa, Artificial Sequence
  inter-sequence between block 1 and block 2
QVPTGG

SEQ ID NO: 40 - protein, 7 aa, Artificial Sequence
  inter-sequence between block 1 and block 2
QVPTGGG

SEQ ID NO: 41 - protein, 8 aa, Artificial Sequence
  inter-sequence between block 1 and block 2
QVPTGGGG

SEQ ID NO: 42 - protein, 9 aa, Artificial Sequence
  inter-sequence between block 1 and block 2
QVPTGGGGG

SEQ ID NO: 43 - protein, 5 aa, Artificial Sequence
  inter-sequence between block 1 and block 2
QVPTA

SEQ ID NO: 44 - protein, 6 aa, Artificial Sequence
  inter-sequence between block 1 and block 2
QVPTAA

SEQ ID NO: 45 - protein, 7 aa, Artificial Sequence
  inter-sequence between block 1 and block 2
QVPTAAA

SEQ ID NO: 46 - protein, 8 aa, Artificial Sequence
  inter-sequence between block 1 and block 2
QVPTAAAA

SEQ ID NO: 47 - protein, 9 aa, Artificial Sequence
  inter-sequence between block 1 and block 2
QVPTAAAAA

SEQ ID NO: 48 - protein, 5 aa, Artificial Sequence
  inter-sequence between block 1 and block 2
  MISC_FEATURE (3)..(3): X is any amino acid
LAXTG

SEQ ID NO: 49 - protein, 6 aa, Artificial Sequence
  inter-sequence between block 1 and block 2
  MISC_FEATURE (3)..(3): X in position 3 can be any amino acid
LAXTGG

SEQ ID NO: 50 - protein, 7 aa, Artificial Sequence
  inter-sequence between block 1 and block 2
  MISC_FEATURE (3)..(3): X in position 3 can be any amino acid
LAXTGGG

SEQ ID NO: 51 - protein, 8 aa, Artificial Sequence
  inter-sequence between block 1 and block 2
  MISC_FEATURE (3)..(3): X in position 3 can be any amino acid
LAXTGGGG

SEQ ID NO: 52 - protein, 5 aa, Artificial Sequence
  inter-sequence between block 1 and block 2
  MISC_FEATURE (3)..(3): X in position 3 can be any amino acid
LAXTG

SEQ ID NO: 53 - protein, 6 aa, Artificial Sequence
  inter-sequence between block 1 and block 2
  MISC_FEATURE (3)..(3): X in position 3 can be any amino acid
LAXTGG

SEQ ID NO: 54 - protein, 7 aa, Artificial Sequence
  inter-sequence between block 1 and block 2
  MISC_FEATURE (3)..(3): X in position 3 can be any amino acid
LAXTGGG

SEQ ID NO: 55 - protein, 9 aa, Artificial Sequence
  inter-sequence between block 1 and block 2
  MISC_FEATURE (3)..(3): X in position 3 can be any amino acid
LAXTGGGGG

SEQ ID NO: 56 - protein, 5 aa, Artificial Sequence
  inter-sequence between block 1 and block 2
  MISC_FEATURE (3)..(3): X in position 3 can be any amino acid
LAXTA

SEQ ID NO: 57 - protein, 6 aa, Artificial Sequence
  inter-sequence between block 1 and block 2
  MISC_FEATURE (3)..(3): X in position 3 can be any amino acid
LAXTAA

SEQ ID NO: 58 - protein, 7 aa, Artificial Sequence
  inter-sequence between block 1 and block 2
  MISC_FEATURE (3)..(3): X in position 3 can be any amino acid
LAXTAAA

SEQ ID NO: 59 - protein, 8 aa, Artificial Sequence
  inter-sequence between block 1 and block 2
  MISC_FEATURE (3)..(3): X in position 3 can be any amino acid
LAXTAAAA

SEQ ID NO: 60 - protein, 9 aa, Artificial Sequence
  inter-sequence between block 1 and block 2
  MISC_FEATURE (3)..(3): X in position 3 can be any amino acid
LAXTAAAAA

SEQ ID NO: 61 - protein, 5 aa, Artificial Sequence
  inter-sequence between block 1 and block 2
  MISC_FEATURE (3)..(3): X in position 3 can be any amino acid
LPXSG

SEQ ID NO: 62 - protein, 6 aa, Artificial Sequence
  inter-sequence between block 1 and block 2
  MISC_FEATURE (3)..(3): X in position 3 can be any amino acid
LPXSGG

SEQ ID NO: 63 - protein, 7 aa, Artificial Sequence
  inter-sequence between block 1 and block 2
  MISC_FEATURE (3)..(3): X in position 3 can be any amino acid
LPXSGGG

SEQ ID NO: 64 - protein, 8 aa, Artificial Sequence
  inter-sequence between block 1 and block 2
  MISC_FEATURE (3)..(3): X in position 3 can be any amino acid
LPXSGGGG

SEQ ID NO: 65 - protein, 9 aa, Artificial Sequence
  inter-sequence between block 1 and block 2
  MISC_FEATURE (3)..(3): X in position 3 can be any amino acid
LPXSGGGGG

SEQ ID NO: 66 - protein, 5 aa, Artificial Sequence
  inter-sequence between block 1 and block 2
  MISC_FEATURE (3)..(3): X in position 3 can be any amino acid
LPXSA

SEQ ID NO: 67 - protein, 6 aa, Artificial Sequence
  inter-sequence between block 1 and block 2
  MISC_FEATURE (3)..(3): X in position 3 can be any amino acid
LPXSAA

SEQ ID NO: 68 - protein, 7 aa, Artificial Sequence
  inter-sequence between block 1 and block 2
  MISC_FEATURE (3)..(3): X in position 3 can be any amino acid
LPXSAAA

SEQ ID NO: 69 - protein, 8 aa, Artificial Sequence
  inter-sequence between block 1 and block 2
  MISC_FEATURE (3)..(3): X in position 3 can be any amino acid
LPXSAAAA

SEQ ID NO: 70 - protein, 9 aa, Artificial Sequence
  inter-sequence between block 1 and block 2
  MISC_FEATURE (3)..(3): X in position 3 can be any amino acid
LPXSAAAAA

SEQ ID NO: 71 - protein, 4 aa, Artificial Sequence
  inter-sequence between block 1 and block 2
  MISC_FEATURE (2)..(4): X in positions 2 to 4 can be any amino acid
NXXX

SEQ ID NO: 72 - protein, 5 aa, Artificial Sequence
  inter-sequence between block 1 and block 2
  MISC_FEATURE (2)..(5): X in positions 2 to 5 can be any amino acid
NXXXX

SEQ ID NO: 73 - protein, 6 aa, Artificial Sequence
  inter-sequence between block 1 and block 2
  MISC_FEATURE (2)..(6): X in positions 2 to 6 can be any amino acid
NXXXXX

SEQ ID NO: 74 - protein, 4 aa, Artificial Sequence
  inter-sequence between block 1 and block 2
  MISC_FEATURE (2)..(4): X is any amino acid
DXXX

SEQ ID NO: 75 - protein, 5 aa, Artificial Sequence
  inter-sequence between block 1 and block 2
  MISC_FEATURE (2)..(5): X in positions 2 to 4 can be any amino acid
DXXXX

SEQ ID NO: 76 - protein, 6 aa, Artificial Sequence
  inter-sequence between block 1 and block 2
  MISC_FEATURE (2)..(6): X in positions 2 to 6 can be any amino acid
DXXXXX

SEQ ID NO: 77 - protein, 5 aa, Artificial Sequence
  inter-sequence between block 1 and block 2
LPNTG

SEQ ID NO: 78 - protein, 6 aa, Artificial Sequence
  inter-sequence between block 1 and block 2
LPNTGG

SEQ ID NO: 79 - protein, 7 aa, Artificial Sequence
  inter-sequence between block 1 and block 2
LPNTGGG

SEQ ID NO: 80 - protein, 8 aa, Artificial Sequence
  inter-sequence between block 1 and block 2
LPNTGGGG

SEQ ID NO: 81 - protein, 9 aa, Artificial Sequence
  inter-sequence between block 1 and block 2
LPNTGGGGG

SEQ ID NO: 82 - protein, 5 aa, Artificial Sequence
  inter-sequence between block 1 and block 2
LPNTA

SEQ ID NO: 83 - protein, 6 aa, Artificial Sequence
  inter-sequence between block 1 and block 2
LPNTAA

SEQ ID NO: 84 - protein, 7 aa, Artificial Sequence
  inter-sequence between block 1 and block 2
LPNTAAA

SEQ ID NO: 85 - protein, 7 aa, Artificial Sequence
  inter-sequence between block 1 and block 2
LPNTAAA

SEQ ID NO: 86 - protein, 8 aa, Artificial Sequence
  inter-sequence between block 1 and block 2
LPNTAAAA

SEQ ID NO: 87 - protein, 9 aa, Artificial Sequence
  inter-sequence between block 1 and block 2
LPNTAAAAA

SEQ ID NO: 88 - protein, 5 aa, Artificial Sequence
  peptidic recognition sequence for sortase
IPKTG

SEQ ID NO: 89 - protein, 5 aa, Artificial Sequence
  peptidic recognition sequence for sortase
APKTG

SEQ ID NO: 90 - protein, 5 aa, Artificial Sequence
  peptidic recognition sequence for sortase
DPKTG

SEQ ID NO: 91 - protein, 5 aa, Artificial Sequence
  peptidic recognition sequence for sortase
SPKTG

SEQ ID NO: 92 - protein, 5 aa, Artificial Sequence
  peptidic recognition sequence for sortase
APATG

SEQ ID NO: 93 - protein, 5 aa, Artificial Sequence
  peptidic recognition sequence for sortase
LPECG

SEQ ID NO: 94 - protein, 5 aa, Artificial Sequence
  peptidic recognition sequence for sortase
  MISC_FEATURE (1)..(1): X in position 1 can be M, L or V
  MISC_FEATURE (2)..(2): X in position 2 can be P, T, A or S
  MISC_FEATURE (3)..(3): X in position 3 can be any amino acid
  MISC_FEATURE (4)..(4): X in position 4 can be A, L, T, S, V or I
XXXXG

SEQ ID NO: 95 - protein, 5 aa, Artificial Sequence
  Inter-sequence between block 1 and block 2
IPKTG

SEQ ID NO: 96 - protein, 5 aa, Artificial Sequence
  Inter-sequence between block 1 and block 2
IPKTA

SEQ ID NO: 97 - protein, 5 aa, Artificial Sequence
  Inter-sequence between block 1 and block 2
APKTG

SEQ ID NO: 98 - protein, 5 aa, Artificial Sequence
  Inter-sequence between block 1 and block 2
APKTA

SEQ ID NO: 99 - protein, 5 aa, Artificial Sequence
  Inter-sequence between block 1 and block 2
DPKTG

SEQ ID NO: 100 - protein, 5 aa, Artificial Sequence
  Inter-sequence between block 1 and block 2
DPKTA

SEQ ID NO: 101 - protein, 5 aa, Artificial Sequence
  Inter-sequence between block 1 and block 2
SPKTG

SEQ ID NO: 102 - protein, 5 aa, Artificial Sequence
  Inter-sequence between block 1 and block 2
SPKTA

SEQ ID NO: 103 - protein, 5 aa, Artificial Sequence
  Inter-sequence between block 1 and block 2
APATG

SEQ ID NO: 104 - protein, 5 aa, Artificial Sequence
  Inter-sequence between block 1 and block 2
APATA

SEQ ID NO: 105 - protein, 5 aa, Artificial Sequence
  Inter-sequence between block 1 and block 2
LPECG

SEQ ID NO: 106 - protein, 5 aa, Artificial Sequence
  Inter-sequence between block 1 and block 2
LPECA

SEQ ID NO: 107 - protein, 5 aa, Artificial Sequence
  Inter-sequence between block 1 and block 2
  MISC_FEATURE (1)..(1): X in position 1 can be M, L or V
  MISC_FEATURE (2)..(2): X in position 2 can be P, T, A, or S
  MISC_FEATURE (3)..(3): X in position 3 can be any amino acid
  MISC_FEATURE (4)..(4): X in position 4 can be A, L, T, S, V or I
XXXXG

SEQ ID NO: 108 - protein, 5 aa, Artificial Sequence
  Inter-sequence between block 1 and block 2
  MISC_FEATURE (1)..(1): X in position 1 can be M, L, or V
  MISC_FEATURE (2)..(2): X in position 2 can be P, T, A, or S
  MISC_FEATURE (3)..(3): X in position 3 can be any amino acid
  MISC_FEATURE (4)..(4): X in position 4 can be A, L, T, S, V, or I
XXXXA

SEQ ID NO: 109 - protein, 7 aa, Artificial Sequence
  peptide sequence bound to resin of fig. 11
GGWTWTW

SEQ ID NO: 110 - protein, 8 aa, Artificial Sequence
  peptide sequence bound to resin of fig. 12
LPETGGRR

SEQ ID NO: 111 - protein, 13 aa, Artificial Sequence
  peptide sequence bound to resin of fig. 13
WTWTWLPETGGRR

(19) United States
(12) Patent Application Publication
(10) Pub. No.: US 2023/0193291 A1
(43) Pub. Date: Jun. 22, 2023

(54) METHOD FOR PRODUCING BLOCKPOYMERS BY MEANS OF LINKING BLOCKS BY A TRANSPEPTIDASE, AND BLOCK POLYMERS OBTAINED BY TRANSPEPTIDASE LINKING

(71) Applicant: FRAUNHOFER-GESELLSCHAFT ZUR FORDERUNG DER ANGEWANDTEN FORSCHUNG E. V., Muenchen (DE)

(72) Inventors: Ulrich Glebe, Potsdam (DE); Xiaolin Dai, Potsdam (DE); Alexander Boeker, Berlin (DE); Martin Schmidt, Berlin (DE); Ulrich Schwaneberg, Kelmis-Hergenrath (BE); Felix Jakob, Erkelenz (DE); Diana Mate, San Sebastian de los Reyes (ES); Tayebeh Mirzaei Garakani, Aachen (DE)

(21) Appl. No.: 16/630,425

(22) PCT Filed: Jul. 10, 2018
(86) PCT No.: PCT/EP2018/068681

§ 371 Date: Jan. 10, 2020

(30) Foreign Application Priority Data
Jul. 11, 2017 (DE) 10 2017 115 522.8

(51) Int. Cl. C12N 15/62; C12N 9/52; C07K 14/435; C08G 69/10

(52) U.S. Cl. CPC C12N 15/62; C12Y 304/22; C12Y 304/2207; C12Y 304/22071; C12N 9/52; C07K 14/43586; C08G 69/10

(57) ABSTRACT

The object of the present invention is a method for the production of block polymers comprising a firstand a second block comprising the method steps:
A) providing a first block having a nucleophilic peptide sequence for afirst transpeptidase enzyme,
B) providing a second block having a peptide recognition sequence for the first transpeptidase enzyme,
C) linking the first block to the second block by means of the first transpeptidase enzyme, wherein the first and second blocks are independently selected from nanoparticles, non-peptide polymers, and recombinant proteins. Such a production method makes it possible to build block polymers from identical or different blocks in a particularly simple and controlled manner.

### CROSS-REFERENCE TO RELATED APPLICATIONS

The present application is a U.S. National Phase of International Patent Application Ser. No. PCT/EP2018/068681 entitled “METHOD FOR PRODUCING BLOCKPOYMERS BY MEANS OF LINKING BLOCKS BY A TRANSPEPTIDASE, AND BLOCK POLYMERS OBTAINED BY TRANSPEPTIDASE LINKING,” filed on Jul. 10, 2018. International Patent Application Ser. No. PCT/EP2018/068681 claims priority to German Patent Application No. 10 2017 115 522.8 filed on Jul. 11, 2017. The entire contents of each of the above-referenced applications are hereby incorporated by reference for all purposes.

### SEQUENCE LISTING

The instant application contains a Sequence Listing which has been submitted electronically in ASCII format and is hereby incorporated by reference in its entirety. Said ASCII copy, created on May 26, 2020, is named Substitute_Sequence_Listing_HEI19301PCTUS.txt and is 30,269 bytes in size.

### TECHNICAL FIELD

Block polymers are a class of substances in which several blocks that have the same or different chemical structureor are made up of the same monomers are bound together.The blocks in the block polymers can in particular also be present in a defined sequence. Such polymers can be synthesized either by sequential polymerization of different monomers or by linking existing blocks with corresponding reactive end groups. However, the synthesis of block polymers is generally difficult and there are only few examples with a high number ofblocks.

### RELATED ART

In the case of block copolymers, where blocks withdifferent structures are linked, it is often impossible orvery difficult to form a certain sequence of blocks.For example, block copolymers of polystyrene and poly(methylmethacrylate) can only be synthesized by anionic polymerization in the order of styrene and then methyl methacrylate. In order to achieve the reverse sequence of the blocks, a complexmulti-5tage procedure had to bedeveloped.

Block copolymers can also be bound together by means of “click chemistry” reactions. For example, the copper- catalyzed azide-alkine cycloaddition and theDiels-Alder cycloaddition are available, in which functionalgroups that are accessible to “click chemistry” can either be incorporated into existing blocks or the blocks can bebuilt up from these functional groups using transitionmetal catalysts. However, a major disadvantage of these “click reactions” is that unspecifically double bonds are bound to each diene (Diels-Alder reactions) or unspecificallyazides and alkynes are linked (azide-alkinecycloaddition).

The subject of this invention is a method for theproduction of block polymers, which is improved with regard to the disadvantages mentioned above. Block polymers which can be produced by this method are the subject offurther independent patent claims.

According to the invention, a method for the production of block polymers is disclosed, comprising a first and asecond block with the method steps:
A) providing a first block having anucleophilic peptide sequence for a first transpeptidase enzyme,
B) providing a second block having a peptiderecognition sequence for the first transpeptidase enzyme,
C) linking the first block to the second block by meansof the first transpeptidase enzyme,
   wherein the first and second blocks are independently selected from: nanoparticles, non-peptide polymers and recombinantproteins.

Such a method according to the invention makes it possible to produce block polymers with different blocks of nanoparticles, non-peptide polymers and recombinant proteins in any sequence as long as the first and second blocks comprise the nucleophilic peptide sequence and thepeptide recognition sequence for a first transpeptidase enzyme. Both the peptide recognition sequence and thenucleophilic peptide sequence may be peptide sequences with several amino acids or, optionally, individual amino acids which aresuitable substrates for the first transpeptidase enzyme. In particular, the first block and the second block can be linked together via the nucleophilic peptide sequence and the peptide recognition sequence, optionally with cleavage of a fragment, in particular from thepeptide recognition sequence. The present invention therefore provides a modular “building block system” for the targeted linking of any blocks as long as the blocks can be provided with the peptide recognition sequences and nucleophilic sequences requiredfor the transpeptidase ligation.

The method according to the invention using atranspeptidase enzyme enables a comprehensive control over the type and sequence of the blocks and is as efficient as the state-of-the-art “click reactions”. In contrast tothe “click reactions”, the inventive enzyme-catalyzed method allows a greater control over the number and sequence of blocks and, in particular, a synthesis of block polymers without theuse of transition metal complexes, suchas, for example, copper complexes. In particular, the method accordingto the invention also allows the production of block polymers from recombinant proteins with a length that is currently not biotechnologically accessible.

At the beginning of the method, thetranspeptidase enzyme recognizes the peptide recognition sequence of the second block and forms an intermediate product between the peptide recognition sequence and an amino acid in the active center of the transpeptidase enzyme, whereby parts of thepeptide recognition sequence, for example, oligopeptides or individual amino acids, can be cleaved off. In a second step, the nucleophilic peptide sequence of the first block can then regenerate the transpeptidase enzyme by nucleophilic attack on the intermediate and release the block polymer from the first and second blocks with a peptide intermediate sequence between the two blocks containing portions of the peptide recognition sequence and thenucleophilic sequence.

According to one variant of the method according to the invention, the second block may comprise a firstfurther nucleophilic peptidesequence.

This first further nucleophilic peptide sequence canbe used to connect the second block with further blocks by means of further transpeptidase enzymes. These transpeptidase enzymes can be the same transpeptidase as the first transpeptidase enzyme described above or can also be transpeptidases with other substrate specificities, in particularwith different peptide recognition sequences compared to the first transpeptidase enzyme.

The first further nucleophilic peptide sequence mayin particular be blocked by a protecting group.

A protecting group can prevent possible cross reactivities between the first further nucleophilic peptide sequenceand the nucleophilic peptide sequence already present inthe first block during the linking of the first blockwith the second block in method step C).

In particular, the first further nucleophilic peptide sequence may also be suitable for the firsttranspeptidase enzyme, the protecting group thenparticularly advantageously blocking the reaction of this first further nucleophilic peptide sequence during method step C). Alternatively, the first further nucleophilic peptide sequence may also be suitable for a transpeptidase enzyme different from the first transpeptidase enzyme.

In particular, already known compounds suchas fluorenylmethoxycarbonyl (Fmoc) and/or tert-butyloxycarbonyl (t-Boc), which can be produced using fluorenyloxymethyl chloride and/or di-tert-butyldicarbonate at the amino or carboxy groups, can also be considered as protecting groups.

A further variant of a method according to the invention is a method for the production of a block polymer with at least three blocks, comprising the further methodsteps:
D) providing a third block having a peptiderecognition sequence for the first transpeptidase enzyme,
E) if present- removing the protecting group ofthe first furthernucleophilic peptide sequence of the second block,
F) linking the third block to the second block bymeans of the first transpeptidase enzyme.

Such a method is particularly suitable forlinking a third block using the same first transpeptidase enzyme. In this case, removing the protecting group of the first further nucleophilic peptide sequence in method step E) ensures that this nucleophilic peptide sequence canbe linked to the peptide recognition sequence of the third block. Such a method is particularly suitable to link the third block to an existing diblock from the first and second blocks in a step-by-Step process, in which the blocks are linked together one after the other in a targeted manner. The thirdblock is preferably added to the reaction solution only after method step E), the cleavage of theprotecting group.

Alternatively, the first further nucleophilic peptide sequence may be different from thenucleophilic peptide sequence present in the first block and can be used in particular for a linkage reaction with asecond transpeptidase different from the first transpeptidase. In such a method, in particular the following further method steps are included:
G) providing a third block having a peptiderecognition sequence for the second transpeptidase enzyme,
H) linking the third block to the second block bymeans of the second transpeptidase enzyme.

In such a method, the advantage is that a second transpeptidase enzyme recognizing a differentpeptide recognition sequence than the first transpeptidase enzyme is used to link the third block to the second block. The different substrate specificity of the second transpeptidase enzyme may prevent that in method step H) portions of the not yet linked second block which may possibly be presentas impurities, instead of the third block, are boundto the growing polymer chain. In some transpeptidase enzymes, especially in some sortases, different sortase enzymes often have a different peptide recognition sequence, but the same nucleophilic sequence.

In such a method, it is also possible to remove a protecting group from the first further nucleophilic sequence of the second block, since such protecting groups also frequently prevent cross-reactions in different transpeptidase enzymes.

The inventive method can also be advantageously modified in such a way that block polymers can be produced with at least four blocks, preferably at least six, more preferablyat least eight blocks. After method steps F)and H) at least one block, preferably three blocks and more preferably five further blocks, each provided with a peptide recognition sequence and a nucleophilic sequence for one ormore transpeptidase enzymes, can be provided and can belinked to each other by means of these one or more transpeptidase enzymes and can be bound to the third block.

In this method variant, transpeptidase enzymes corresponding either to the first and/or second transpeptidase enzyme or having different substrate specificities from these two transpeptidase enzymes may be used. In such a method,too, protecting groups may again be present at thenucleophilic sequences to prevent unwanted reactions.

According to a further embodiment of a method according to the invention, the method can be used for the productionof block copolymers in which blocks with different structures are used for at least two blocks of the first, secondand optionally present third and further blocks.

In such a method, blocks with different chemical structures can be linked together in a targeted manner without the limitations of the state of the art regarding the sequenceof the blocks. For example, two different polymerblocks, such as polyethylene glycols or derivatives of polyethylene glycols and polyacrylamides or their derivatives, can be linked together in a targeted manner.

In particular, the inventive variants ofthe production method can be used tolink together completely artificial blocks such as synthetic polymers (plastics) or small particles such as nanoparticles. Possible examplesof polymers are polymers made up of vinyl monomers, for example: styrene and derivatives, vinylpyridines, acrylic acid and acrylic acid esters, methacrylic acid and methacrylic acid esters, acrylamides, N-Substituted and N, N-disubstituted acrylamides, dienes, diacrylamides, dimethacrylates, acrylonitrile, 1-vinylimidazole andmaleic anhydride.

It is also possible to link non-peptide biopolymers suchas polyhydroxybutyrates, cellulose, chitin,starch, polylactides, carbohydrates and combinationsthereof. Such biopolymers as blocks can also be easily linked with synthetic polymers such as plastics.

However, peptide biopolymers, in particular recombinant proteins, can also be used for the first, second and possibly existing third and further blocks. The peptide biopolymers can be expressed particularly in cloning vectors in such a way that they already have the peptide recognition sequence and optionally also the nucleophilic sequence. In particular, the peptide recognition sequence may be present at the c-terminus of the recombinant proteins and the nucleophilic sequence at the N-terminus.

The peptide biopolymers may in particular be selected from structural proteins such as, for example, elastin, fibroin, sericin, collagen and keratin and combinations thereof. Particularly preferred are silk proteins such as, for example, spider silk proteins, which exhibit exceptional mechanical resilience in relation to their weight,in particular elasticity and tear resistance.Recombinant silk proteins, such as, for example, spider silk proteins, can often not be expressed in the same length as natural spider silk proteins, but merely shortened. However, sincethe mechanical resilience increases with the length of thesilk proteins, it is particularly advantageous to link blocks of recombinant silk proteins with each other by means of the method according to the invention and thus to obtain longer silk protein aggregates with improved mechanical properties. All recombinantly available fragments or variants of silk proteins can be used as silk proteins. In particular, the recombinant spider silk proteins described in PCT patent application WO 2006/008163 A2 can be linked with each other. This PCT patent application is hereby referred toin full with regard to the spider silk proteins.

According to a further embodiment of a production method according to the invention, this method can in particular also be used for the production of branched block polymers. At least one of the first, second and optionally existing third and further blocks comprises at least three sequences selected from the peptide recognition sequences and nucleophilic sequences for a transpeptidase enzyme tobind further blocks. Blocks with at least threesequences for binding further blocks by means of transpeptidase enzymes can, for example, be used for the production of graft polymers and graft copolymers as well as for the production of star-Shaped bonds. Nanoparticles, such as Si02 nanoparticles, can also be provided with a largenumber of sequences for binding further blocks, so that these particles can, for example, have layers of block polymers on their surface that have been bound via transpeptidase enzymes.

The transpeptidase enzymes that can be used are in particular sortases or their fragments, for example Sortase A, Sortase B, Sortase C, Sortase D, Sortase E and other transpeptidase enzymes such as, for example, butelase and/or trypsiligase.

In particular, a soluble, catalytic domain of thesortase enzyme Sortase A SrtA of Staphylococcus aureus can be expressed recombinantly (Belscher, J. G., et al. (2011) Sortase A as a tool for high-yield histatin cyclization. FASEB J. 25, 2650-2658). This transpeptidase enzyme recognizes the peptide recognition sequence—LPXTG (SEQ ID NO:1),where amino acid X represents any amino acid, e.g.—LPETG (SEQ ID NO:2) or —LPATG (SEQ ID NO:3). Other variants of the sortase are, for example, Sortase B srtB of Staphylococcus aureus or Bacillus anthracis, which have a peptide recognition sequence—NP(Q/K)TN (SEQ ID NO: 4) other than srtA (Maressa, A. W. et al. J. Bacterial. 2006, 188,8145-8152 and Mazmanian, S. Ket al. o. Proc. Natl. Acad. Sci. U.S.A. 2002, 99, 2293-2298). Further C, D and E class sortases with different peptide recognition sequences are also known (Spirig, T. et al. Mol. Microbial. 2011, 82, 1044 and Bradshaw, W. J.; et al. FEBS J. 2015, 282, 2097). Class C and D sortases comprise the peptide recognitionsequences—(I/L)(P/A)XTG (SEQ ID NO:5) (class C) and —QVPTG (SEQ ID NO:6) or —LPNTA (SEQ ID NO: 7) (class D) and class E sortases comprise the recognition sequence—LAXTG (SEQ ID NO: 8). Sortase A can generally recognize a whole series of peptide recognition sequences of the general sequence-(M/L/V)(P/T/A/S)X(A/L/T/S/V/I)G (SEQ ID NO:9), where X againstands for each amino acid, in particular A (Ning Li et al., Biochem Biophys Res Commun, 2017, 486 (2), 257-263). Other peptide recognition sequences recognized by sortases are IPKTG (SEQ ID NO: 88), APKTG (SEQ ID NO:89), DPKTG (SEQ ID NO: 90), SPKTG (SEQ ID NO: 91), APATG (SEQ ID NO: 92) and LPECG (SEQ ID NO: 93) (John M. Antos et al., Curr. Opin. Struct. Biol.; 2016, 38:111-118).

Butelase 1, an Asn/Asp(Asx) peptide ligase, may also be used instead of or in addition to the sortases (G. K. T. Nguyen, et al. Nat. Chem. Biol., 2014, 10, 732-738). This transpeptidase enzyme has the peptide recognition sequence -NHV or -DHV. In addition, trypsiligase can be used alternatively or additionally as transpeptidase, whichhas the peptide recognition sequence andthe nucleophilic sequence Y-RH, where Y may be present, but does not have to be present. During the formation of the intermediate product between the transpeptidase and the peptide substrate, Y and RH are cleaved if Y is present, resulting in an intermediate sequence -YRH-between two blocks linked by the trypsiligase.

In methods according to the invention, the peptide recognition sequences can therefore be independently selected from the followingsequences: -LPXTG (SEQ ID NO. 1), - LPXTA (SEQ ID NO:10), -NPQTN (SEQ ID NO: 4), -QVPTG (SEQ ID NO: 6), -LAXTG (SEQ ID NO:8), LPXSG (SEQ ID NO: 12), -NHV, -DHV, -(Y)zRH and -(M/L/V)(P/T/A/S)X(A/L/T/SN/I)G (SEQ ID NO: 94) ,IPKTG (SEQ ID NO: 88), APKTG (SEQ ID NO:89), DPKTG (SEQ ID NO: 90), SPKTG (SEQ ID NO: 91), APATG (SEQ ID NO: 92) and LPECG (SEQ ID NO: 93), wherein X is any possible amino acid and the parameter z may be O or 1.

The recognition sequences depend on the classes of transpeptidase enzymes, in particular the classes of sortases andbutelase.

The nucleophilic sequences may beselected independently from: -(G)₁₋₅ (SEQ ID NOs: 13-14), preferably -(G)₁₋₃, and -(A)₁₋₅ (SEQ ID NOs: 15-16).

It should be noted that the butelase can recognize any amino acids as nucleophilic sequences, and thus their nucleophilic sequence can be called -(X)₁₋₅-. In the case of the trypsiligase, the nucleophilic sequence is Y-RH. The different classes of sortase enzymes recognize different peptide recognition sequences, but have the same nucleophilic sequence -(G)₁₋₅ (SEQ ID NOs: 13-14), preferably -(G)₁₋₃, or also -(A)₁₋₅ (SEQ ID NOs:15-16).

The catalytic mechanism of asortase-catalyzed reaction begins with the cleavage of the last amino acid of the peptide recognition sequence and the linking of the remaining peptide recognition sequence to a reactive cysteine residue in the active center of the sortase enzyme. The thioester-acyl intermediate can now be nucleophilicallyattacked by the N-terminal amino acid of a nucleophilic sequence containing an oligoglycine or alanine motif for most sortases and which can be any amino acid in butelase. This forms a peptide bond between the two substrates, the polymer blocks with parts of the peptide recognition sequence andthe nucleophilic sequence as peptide intermediate sequence and regenerates the sortase enzyme. This peptide intermediate sequence contains the aforementioned peptide recognition sequence without the amino acids cleaved off during the catalytic reaction, whereby the remaining peptide recognition sequence being linked to the nucleophilic sequence.

Nucleophilic sequences comprising between 1 and 5 glycines and between 1 and 5 alanines are particularly suitable as nucleophilic groups for attacking the thioester-acyl intermediates from the sortase enzyme and the peptide recognition sequence block and thus releasing the reaction product, the blocks linked by peptide intermediate sequences and the sortaseenzyme.

Since the transpeptidase enzymes, in particular the sortases, catalyze an equilibrium reaction between the transpeptidation, the formation of a bond between the block with the peptide recognition sequence and the block withthe nucleophilic sequence, and the dissolution of this bond to form the starting blocks, measures can be taken toshift the equilibrium of the reaction to the transpeptidation side. This can be ensured in particular by irreversibly removing from the equilibrium reaction the amino acids or oligopeptides cleaved off between the two blocks during the formation of the peptide bond. For example, behind theamino acid that is cleaved off from the peptiderecognition sequence during the sortase reaction, which is often a glycine, a histidine can be bound, so that the cleaved oligopeptide can then be completely removed from the equilibrium by, for example, complexation with Ni²⁺, Co²⁺, Cu²⁺ or Fe²⁺.

Alternatively or additionally, both the sequence of the peptide recognition sequence and the nucleophilic sequence can be constructed insuch a way that after the ligation by the transpeptidase enzyme they together form a betahairpin structure which cannot be attacked by the transpeptidase, so that the equilibrium of the reaction is shifted in the direction of transpeptidation. Such beta hairpin structures can, for example, be formed by the sequence—WTWTW-(SEQ ID NO:17), which can be integrated into both the peptide recognition sequence and the nucleophilic sequence.

Another possibility is to design the amino acid residues of the peptide recognition sequence, which are cleaved off during ligation, as a weak nucleophile, so that the reverse reaction, the cleavage of the two blocks, isalso not favored. Such nucleophilic groups can, for example, be present at the C-terminus of the peptide recognition sequence so that they form a weak nucleophile after cleavage (see e.g. FIG. 5).

The expression of the transpeptidase enzyme, for example, the sortase, can preferably take place in solubleform, for example, without a transmembrane domain and with a His-tagat the N-terminus. Escherichia coli, for example, is apreferred expression system that can recombinantly express proteins in high yields. The purification of the transpeptidaseenzyme can then be carried out using Ni2+affinity chromatography over the His-tags.

The linking of the individual blocks with the peptide recognition sequences and the nucleophilic sequencesusing transpeptidase enzymes can then be carried out in vitroin a reaction vessel in buffered solution. For example, a buffer can be used which contains between 40 and 60 mM of a buffer with a pH between 6.8 and 7.8, preferably 7.5, to ensure the enzymatic activity of the transpeptidase enzyme, forexample, Tris-HCl. The buffer solution may also containbetween 100 and 200 mM of an alkali metal halide, for example, NaCl, and about 4 to 8 mM of an alkaline earth metal halide, for example, CaCl2. The reaction can be carried out at temperatures between 25-40° C., preferably between 28° C and 37° C. For a linkage reaction between a block witha peptide recognition sequence and a block with a nucleophilic sequence, both blocks can be used, for example, in equimolar amounts. However, one reaction partner can also be used in molar excess, for example, to shift the equilibrium ofthe linkage reaction strongly in the direction ofthe linkage reaction. In particular, the block with the nucleophilic sequence can be used in molar excess of, for example, upto 50:1 compared to the block with the peptide recognition sequence for this purpose. As far as the catalytic activity of the transpeptidase enzymes is concerned, in the case of Sortase A, sortases with a catalytic efficiency in the range of k_{cat}/Kₘ [Abz-LPETGK(Dnp)-CONH₂]=(100- 50 000) M⁻¹ s⁻¹, and especially preferably between k_{cat}/Kₘ [Abz-LPETGK(Dnp)-CONH₂]=(180- 35 000) M⁻¹ s⁻¹ can be used, the activities being measured on asubstrate Abz-LPETGK(Dnp)-CONH2having the fluorophore 2-aminobenzoic acid (Abz) as a marker at the N-terminus and at lysine 2,4-dinitrophenyl (DNP). The catalytic efficiencies of other sortase enzymes may be lower than the efficiencies for Sortase A orcomparable.

Furthermore, the starting block with which the method forthe production of the block polymers is initiatedmay be immobilized on a solid phase, for example beads, such as polystyrene beads, in order to make a purification between the individual reaction steps particularly easy. In particular, purification enables the removal ofunlinked and unreacted blocks with peptide recognitionsequences and/or nucleophilic sequences by purification, so that they do not lead to undesired side reactions during the next linking step. In the production of block polymers, for example, polystyrene beads linked via divinylbenzene with a loadin the range of 0.2-1 mmol/g to functional groups canbe used for binding. The blocks are bound, for example, by an ester bond to a 4-alkoxybenzyl alcohol or by an amide bond to a 4- alkoxybenzyl-oxycarbonylhydrazide. The finished block polymers can be separated from the polystyrene beadsby trifluoroacetic acid without affecting the peptide bonds in the block polymers. In the second group, a peptide amideis formed during cleavage.

By means of the methods according to the invention, block copolymers in particular can be produced which have hydrophilic blocks next to hydrophobic blocks or hydrophobic areas next to hydrophilic areas. Thus, forexample, so-called “crew-cut” micelles can also be produced in which hydrophobic block areas, for example, protein areas, are present in the core of the micelles and the hydrophilic areas of the blocks protrude outwards into a hydrophilic, for example, aqueous medium.

The subject of this invention is also a method forthe production of block polymers with the methodsteps:
A1) providing a plurality of blocks having a nucleophilic sequence and a peptide recognition sequence for atleast one transpeptidase enzyme,
B1) linking the plurality of blocks by the at leastone transpeptidase enzyme,
   wherein the plurality of blocks is independently selected from: nanoparticles, non-peptide polymers,and recombinant proteins.

For the purpose of the present invention, a “plurality of blocks” means at least three blocks. By means of themethod according to the invention, anynumber of blocks, preferably between 3 and 50, more preferably at least three to 30 blocks, most preferably between 4 and 10 blocks, can be linked together. In the case of spider silk proteins, up to5 blocks in particular can be linked together. With regard to this inventive method variant, all features already described above also apply. When structural proteins are used asblocks which contain repeat units of protein sequences, the number of blocks linked by the methods according to the invention can be reduced with an increasing number of repeat units per block without significantly reducing the total length ofthe resulting blockpolymers.

In particular, in such a method variant, the nucleophilic sequence may be blocked by a protecting group in at least some, preferably all, of the plurality of blocks, whereby the protecting groups are then removed in method step Bl) before linking.

The protecting groups block the nucleophilic sequences that are not yet supposed to react in the respective linking step, as already describedabove.

According to a further embodiment of the method according to the invention, the plurality of blocks can be linked in method step Bl), step by step and block by block.

Such a method allows a particularly controlledblock-by-block synthesis of block polymers and inparticular allows the production of block copolymers with a defined sequence of blocks. Such a control over the linking of the blocks is often not possible or very difficult with conventional synthesis methods for block copolymers.

Alternatively, in method step Bl), the plurality of blocks can also be linked in a one-pot method.

With such a method, it is particularly easy to link all blocks with nucleophilic sequences andpeptide recognition sequences by a transpeptidase present in the reaction solution in a reaction vessel using one reaction step. Such a one-pot method can be particularly advantageous if block polymers are to be produced which do not have tohave a defined sequence of blocks and/or which can have asize distribution without a defined size.

With all methods according to the invention, itis also particularly easy to link blocks with different peptide recognition sequences for different transpeptidase enzymes simultaneously in only one method step, as long as the different transpeptidase enzymes required for this are made available simultaneously in the reaction solution andas long as these different transpeptidase enzymes also preferably have different nucleophilic sequences. For example, blocks with several linked peptide recognition sequences for different transpeptidase enzymes can be used as starting compounds to which further blocks can then be coupledin a single step using different transpeptidases.

The present invention also provides block polymers with at least a first block 1 and a second block 2 with the following structure:

block 1- Peptide intermediate sequence 1- block 2 wherein the peptide intermediate sequence 1 bridging block 1 and block 2 contains a sequence or is a sequence selected from the following group:
-(I/L)(P/A)XT(G)₁₋₅-(SEQ ID NOs: 18-22), -(I/L)(P/A)XT(A)₁₋₅-(SEQ ID NOs:23-27), -NP(Q/K)T(G)₁₋₅-(SEQ ID NOs: 28-32), -NP(Q/K)T(A)₁₋₅-(SEQ ID NOs:33-37), -QVPT(G)₁₋₅-(SEQ ID NOs: 38-42), -QVPT(A)₁₋₅-(SEQ ID NOs: 43-47), -LAXT(G)₁₋₅-(SEQ ID NOs: 48-52), -LAXT(A)₁5-(SEQ ID NOs: 56-60), -LPXS(G)₁₋₅-(SEQ ID NOs: 61-65), -LPXS(A)₁₋₅-(SEQ ID NOs: 66-70), -N (X)₁₋₅-(SEQ ID NOs:71-73), -D(X)₁₋₅-(SEQ ID NOs: 74-76),
-LPNT(G)₁₋₅-(SEQ ID NOs: 77-81), -LPNT(A)₁₋₅-(SEQ ID NOs:82-87), IPKT(G)₁₋₅ (SEQ ID NO: 95, IPKT(A)₁₋₅ (SEQ ID NO:96), APKT(G)₁₋₅ (SEQ ID NO: 97), APKT(A)₁₋₅ (SEQ ID NO: 98), DPKT(G)1-5 (SEQ ID NO: 99), DPKT(A)1-5 (SEQ ID NO: 100),SPKT(G)1-5 (SEQ ID NO: 101), SPKT(A)₁₋₅ (SEQ ID NO: 102), APAT(G)₁₋₅ (SEQ ID NO: 103), APAT(A)₁₋₅ (SEQ ID NO: 104) and LPEC(G)₁₋₅ (SEQ ID NO: 105), LPEC(A)₁₋₅ (SEQ ID NO: 106), -(M/L/V) (P/T/A/S)X(A/L/T/S/V/I) (G)1-5 (SEQ ID NO: 107) and -(M/L/V) (P/T/A/S)X(A/L/T/S/V/I) (A)₁₋₅ (SEQ ID NO: 108), in particular -LPXT(G)₁₋₅-(SEQ ID NO: 1), -LPXT(A)₁₋₅-(SEQ ID NO: 10), -NPQT(G)₁₋₅-(SEQ ID NO: 28), -NPQT(A)₁₋₅-(SEQ ID NO: 4), wherein X is any possible amino acid,
   and wherein block 1 and block 2 are independently selected from:

nanoparticles, non-peptide polymers,and recombinant proteins.

The sequence of the peptide intermediate sequence 1 runsfrom N-terminus to C-terminus as usual for protein sequences.The intermediate sequence 1 is composed of parts ofthe peptide recognition sequence, whereby at least one amino acid, usually glycine, is cleaved off from the C-terminus of the intermediate sequence during the transpeptidase-catalyzed reaction and the residue of the peptide recognition sequence is linked to the nucleophilic sequence (frequentlyglycine or alanine) The peptide intermediate sequence 1 describedabove is therefore built up starting from the N-terminus fromparts of the peptide recognition sequence linked to the nucleophilic sequence, wherein the intermediate sequences1 described above contain parts of thepeptide recognition sequences of the already known Sortases A to E. The short peptide intermediate sequences -N(X)1-5-(SEQ ID NOs: 71-73), -D(X)1-5-(SEQ ID NOs: 74-76) correspond to the intermediate sequences expected from a butelase- catalyzed ligation when the dipeptide -HV is cleaved offfrom the peptide recognition sequence of the butelase.

This peptide intermediate sequence 1, as well as the intermediate sequences described below, can also be part ofa longer peptide sequence between the individual blocks.In particular, further peptide sequences orother chemical groups may be present as spacers between these sequencesand the blocks bridged bythem.

Block polymers according to a further aspect of the present invention may additionally comprise a third block 3 andthus have the following structure:
block1—peptide intermediate sequence 1-block 2—peptide intermediate sequence 2-block 3
   wherein the peptide intermediate sequence 2 independently of the peptide intermediate sequence 1 contains a sequence or is a sequence selected from the following group:
-(I/L)(P/A)XT(G)₁₋₅-(SEQ ID NO: 18-22), -(I/L)(P/A)XT(A)₁₋₅-(SEQ ID NOs:23-27), -NP(Q/K)T(G) ₁₋₅-(SEQ ID NOs: 28-32), -NP(Q/K)T(A)₁₋₅-(SEQ ID NOs:33-37),-QVPT(G)₁₋₅-(SEQ ID NOs: 38-42), -QVPT(A)₁₋₅-(SEQ ID NOs: 43-47),-LAXT(G)₁₋₅-(SEQ ID NOs: 48-52), -LAXT(A)₁₋₅- -(SEQ ID NOs: 56-60), -LPXS(G)₁₋₅-(SEQ ID NOs: 61-65) , -LPXS(A)₁₋₅-(SEQ ID NOs: 66-70), -N(X)₁₋₅-(SEQ ID NOs:71-73), -D(X)₁₋₅-(SEQ ID NOs: 74-76), -LPNT(G)15-(SEQ ID NOs: 77-81) ,-LPNT(A)₁₋₅-(SEQ ID NOs:82-87), IPKT(G)₁₋₅ (SEQ ID NO: 95), IPKT(A)₁₋₅ (SEQ ID NO:96), APKT(G)₁₋₅ (SEQ ID NO: 97), APKT(A)₁₋₅ (SEQ ID NO: 98), DPKT(G)₁₋₅ (SEQ ID NO: 99), DPKT(A)₁₋₅ (SEQ ID NO: 100), SPKT(G)₁₋₅ (SEQ ID NO: 101), SPKT(A)₁₋₅ (SEQ ID NO: 102), APAT(G)₁₋₅ (SEQ ID NO: 103), APAT(A)₁₋₅ (SEQ ID NO: 104) and LPEC(G)₁₋₅ (SEQ ID NO: 105), LPEC(A)₁₋₅ (SEQ ID NO: 106),
(M/L/V) (P/T/A/S)A(A/L/T/S/V/I)(G)1-5 (SEQ ID NO: 107) and -(M/L/V)(P/T/A/S)A(A/L/T/S/V/I)(A)₁₋₅ (SEQ ID NO: 108), in particular -LPXT(G)1-5-(SEQ ID NO: 1),-LPXT(A)₁₋₅-(SEQ ID NO: 10), -NPQT(G)₁₋₅-(SEQ ID NO: 28), and-NPQT(A)₁₋₅-(SEQ ID NO: 4),
   and wherein block 1, block 2 and block 3 are independently selected from:

nanoparticles, non-peptide polymers, andrecombinant proteins.

By further transpeptidase-catalyzed reactions, the block polymers of the present invention can be build up block by block, wherein between each two blocks different or identical intermediate sequences can be present, depending on which transpeptidase enzyme was used for the respective linkage.

In general, additional repeat units of the generalstructure:

[-peptide intermediate sequence-block ] with the number n can be linked together by methods according to theinvention, resulting in block polymers with the following general structure:
block 1-peptide intermediate sequence 1-block 2-peptide intermediate sequence 2-block 3-[peptide intermediate sequence- block]ₙ,
   wherein then additional peptide intermediate sequences are independently selected from the corresponding group already described above, and wherein then additional blocks are independently selected from the corresponding group already described above, and - wherein n is an integer between 1 and 50, preferably between 3 and 30, most preferably between 4 and 10.

Block polymers and, in the presence of blocks of different chemical structure, also block copolymers with large, defined lengths can be produced by means of the methods according to the invention. In particular, as already described above, the blocks can be non-peptide polymers, which can be independently selected frompoly(methyl methacrylates), polyethylene glycols, acrylamides, and the polymers already described above, which are composed of vinyl monomers, or can also be selected from recombinant proteins or nanoparticles. In particular, it is also possible to couple identical or different fragments of a recombinant spider silkprotein one behind the other as blocks to achieve a particularly high mechanical stability.

Block polymers and block copolymers produced by methods according to the invention can be used for a variety of applications, for example, as polymer-based actuators, as components for solar cells, as materials for medical diagnostics and drug transport, for organic light-emitting diodes (OLEDs), in microelectronics or asmultifunctional plastic materials.

For example, in the case of polymer-based actuators,for example in automotive engineering, a possible blockcan be made of silicone or isoprene and another block of poly(2-vinylpyridine). In order to achieve a sufficient dielectric effect, the molecular weight distribution of the synthesized block copolymers must be narrow, which is accomplished particularly well using the production methodsaccording to theinvention.

For example, blocks comprising itaconic acid polymers, or polyelectrolytes such as polyvinylpyridine, and/or glucose methacrylate polymers can also be coupled to metallic nanoparticles by means of the production methods accordingto the invention, whereby these polymers become conductive.

The possibility of linking polymers, in particular alsonon-peptide polymers and biopolymers, to nanoparticles by means of various variants of methods according to the invention allows to achieve a high density of polymer blocks per nanoparticle. At the same time, the blocks to be offeredcan be provided individually in terms of structure and lengthin the production methods according to the invention.

The different production methods according to the invention as well as the different variants of block polymers and block copolymers according to the invention are also characterized by the fact that, in contrast to many conventional production methods, production takes place without transition metal catalysts and therefore the final produced block polymers and block copolymers are free of transition metal catalysts.In conventional production methods, these transition metal catalysts often have to be removed with great effort,which is particularly demanding for biologicalpolymers.

### BRIEF DESCRIPTION OF THE FIGURES

In the following, aspects of the present inventionwill be explained in more detail on the basis of exemplary embodiments andFigs.:

FIG. 1 shows various aspects of productionmethods according to the invention in which nanoparticles, non- peptide polymers and recombinant proteins can each be linked together.

FIG. 2 shows a variant of a method according tothe invention either for blockwise targeted constructionor by means of a one-pot reaction of block polymers and block copolymers, containing non-peptide polymers, as well as recombinant proteins, which for example can also imitatea structure occurring in nature(bio-mimetic proteins).

FIG. 3 shows a possibility to provide non-peptidepolymer blocks with end groups containing the peptide recognition sequences and nucleophilic sequences for thetranspeptidase enzymes.

FIG. 4 shows in detail a possibility of a stepwise block polymer synthesis with nucleophilic peptide sequenceswhich are provided with protectinggroups.

FIG. 5 schematically shows various possibilitiesto shift the equilibrium reaction of a transpeptidase reaction inthe direction of linkingblocks.

FIG. 6 shows a diagram of a MALDI-ToF mass spectrum of reaction mixtures of linking reactions betweennanoparticles and polymers as blocks produced according to a method according to the invention and of MALDI-ToF mass spectraof negative controls.

FIG. 7 shows transmission electron microscopy (TEM) images of nanoparticle polymer hybrid particles produced as block copolymers according to a method according to the invention.

FIG. 8 shows a MALDI-ToF mass spectrum of blocks with peptide recognition sequences and blocks with nucleophilic sequences, wherein the blocks are eachnon-peptide polymers. Furthermore, the mass spectrum of the reaction product,the block copolymer, is shown.

FIG. 9 shows the design of polymer-peptide (left) and peptide-polymer (right) building blocks fortranspeptidase- mediated ligation. The arrow shows the direction ofpeptide synthesis by solid phase peptidesynthesis.

FIG. 10 shows the strategy for the synthesis of peptide polymer building blocks using two N-terminal glycines andthe polymer poly(N-isopropyl acrylamide) (PNIPAM) asexamples. The grey spheres represent the resin ofthe peptide synthesis.

FIG. 11 shows the synthesis strategy for peptidepolymer (B) building blocks using the example of several N-terminal amino acids and the polymer poly(dimethylaminoethyl methacrylate) (PDMAEMA). The grey spheres symbolize theresin of the peptide synthesis. The Fmoc protecting group at theN- terminus is not shownhere.

FIG. 12 outlines the strategy for the synthesis of polymer- peptide building blocks using the example of a possible recognition sequence and the polymer PNIPAM. The grey spheres symbolize the resin of the peptide synthesis.

FIG. 13 illustrates the synthesis strategy for polymer peptide (A) building blocks with a possible recognition sequence, supplemented by amino acids for the formation of a hairpin structure, and the polymer PNIPAM. The grey spheres symbolize the resin of the peptide synthesis.

### DETAILED DESCRIPTION

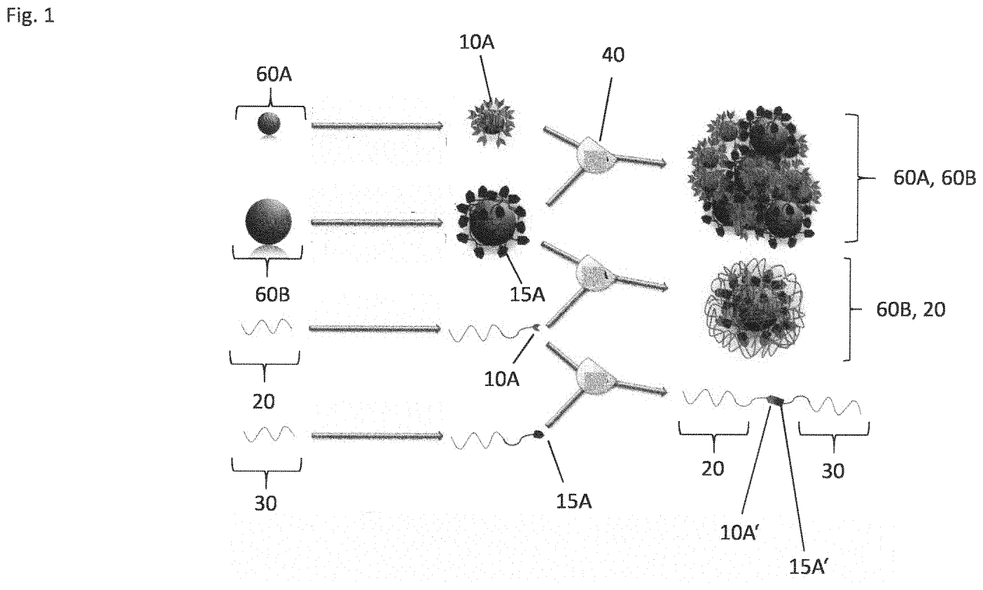

FIG. 1 shows schematically different embodiments of methods according to the invention. The upper part shows how small nanoparticles 60A, for example with a diameter of 60 nm, are provided with C═C double bonds via a functionalization reaction and then provided with a nucleophilic peptide sequence 10A on their surface. These particles, comprising nanoparticles coupled to a nucleophilic peptide sequence, can now be coupled with larger nanoparticles 60B provided with peptide recognition sequences 15A using a transpeptidase enzyme 40. Similar to the small nanoparticle 60A, the peptide recognition sequences can also be provided with C═C double bonds by functionalizing the nanoparticle surface, whichcan then couple with the nucleophilic peptide sequence, for example, by means of a “thiol-click reaction”.

Furthermore, non-peptide polymers 20, for example polyethylene glycol, can be modified with a nucleophilic peptide sequence 10A. Similarly, other non-peptide polymers 30, such as poly(methylmethacrylates), can also be provided with peptide recognition sequences 15A. These non-peptide polymers can then be linked together by a reaction catalyzed by a sortase 40 and form a block copolymer with the polymer blocks 20 and 30, whereby a peptide intermediate sequence 10A′, 15A′ is formed between both polymer blocks. Similarly, the non-peptide polymer blocks with the peptide recognition sequences can also be coupled with nanoparticles to form hybrid materials of nanoparticles and non-peptide polymers 60B, 20.

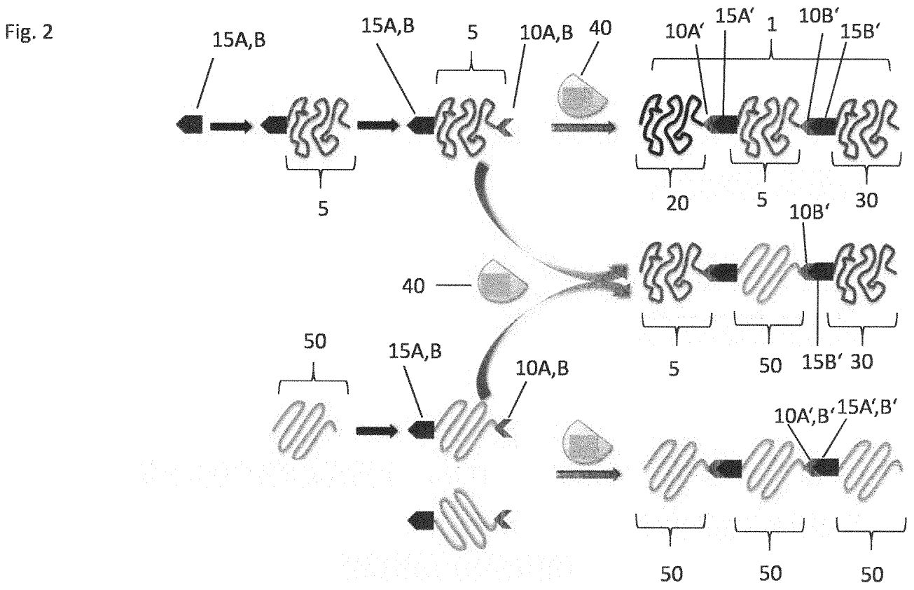

FIG. 2 schematically shows a synthesis of block polymers and block copolymers, wherein the synthesis iscarried out step by step for the individual polymer blocks or in a one-pot reaction. In the upper part of the Fig. a non-peptide polymer 5 with a peptide recognition sequence 15A,B is shown. The reference number 15A,B indicates that it can be either the peptide recognition sequence 15A fora first transpeptidase enzyme or the peptide recognition sequence 15B for a second enzyme different from the first transpeptidase enzyme. Furthermore, this polymer block 5 also has a nucleophilic peptide sequence 10A,B which can be used either by a first transpeptidase enzyme as sequence 10Aor by a second transpeptidase enzyme as sequence lOB as nucleophile. By means of a transpeptidase (sortase), this non-peptide polymer block 5 can be coupled step by step with further polymer blocks. For example, this polymer block 5 can be linked with a further polymer block 20, for example also a non-peptide polymer block, via the peptide recognition sequence 15A of block 5. In this case the polymer block 20 has a nucleophilic peptide sequence 10A for a first sortase enzyme. After ligation, a peptide intermediate sequence 10A′ and 15A′ is present between blocks 20 and 5.Via the nucleophilic peptide sequence lOB for a second sortase enzyme, the polymer block 5 can also be coupled with a further polymer block 30, which has a peptide recognition sequence 15B for the second sortase enzyme. After ligation, another peptide intermediate sequence 10B′ and 15B′ is present between both blocks 5 and 30.

In the middle and lower part of FIG. 2 it is shown how the non-peptide polymer blocks 5 and 30 can also be combined with polymer blocks 50, which contain recombinant proteins. These recombinant proteins can, for example, imitate naturally occurring functions of proteins, such as, for example, recombinant silk proteins such as spider silk proteins and can therefore be labelled as biomimetic molecules. FIG. 2 shows that the transpeptidase enzymes 40 can be used to combine the non-peptide polymer blocks 5 and 30 with protein polymer blocks or to form block polymers consisting exclusively of protein blocks 50.

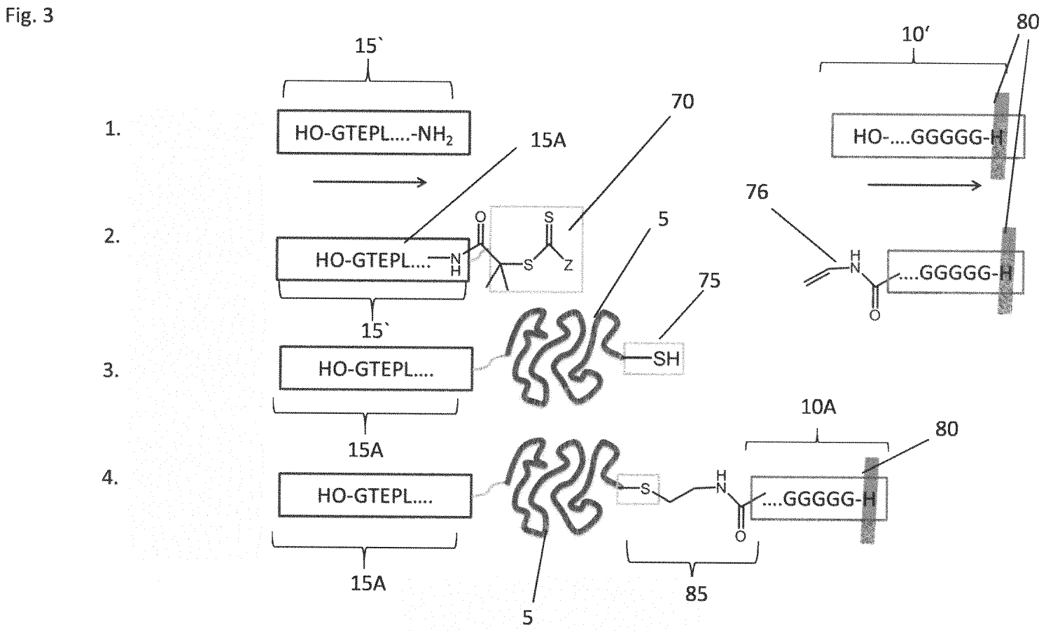

FIG. 3 schematically shows a possibility of how non-peptide polymer blocks can be provided at their ends with nucleophilic peptide sequences andpeptide recognition sequences for the transpeptidase enzymes.

First, the peptide recognition sequences 15′ and the nucleophilic sequences 10′ are produced from the C-terminus to the N-terminus using peptide synthesizers. The production is shown schematically under point II 1.11 in FIG. 3, wherein the protein sequences shown there run analogously to the synthesis from the C-terminus to the N-terminus. In a second step, designated with 11 2.11 , an initiator for e.g. controlled radical polymerization can be attached to the peptide recognition sequence 15′ or so-called RAFT CTAs 70 can be generated on the peptide recognition sequence (RAFT: reversible addition fragmentation chain transfer, CTA: chain transfer agent). As shown under point “2”, the nucleophilic peptide sequence 10′ can be produced separately on apeptide synthesizer, wherein a double bond 76 can be inserted asend group. This end group with a double bond can often be attached to the C-terminus, especially afterpeptide synthesis. This nucleophilic peptide sequence alsocontains a protecting group 80, which is supposed to later block unwanted reactions of the nucleophilic sequence during coupling with other blocks.

Starting from this group with the double bond, a growing polymer chain can be generated at the peptide recognition sequence, for example, by controlled radical polymerization (CRP), as shown under point “3.”. This polymerization can be done by ATRP (ATRP: atom transfer radical polymerization)and RAFT techniques, in which a large part of thegrowing radical chain is converted into a “sleeping” form and thus chain termination reactions can be minimized. By controlling radical polymerization, polymer blocks with a narrow molecular weight distribution can be synthesized. At theend of the polymer chain, the CTA group can be converted into a -SH group 75. As shown under point “4.”, the nucleophilic sequence 10A can be linked via a so-called “thiol click chemistry” via a reaction of the -SH group 75 with the double bond 76 of the nucleophilic peptide group via the group85 formed thereby so that a polymer block 5 is formed, at the ends of which a peptide recognition sequence 15A and a nucleophilic peptide sequence 10A with a protecting group80 are present. Polymer blocks with recombinant proteins as blocks can, for example, be produced particularly easilyby cloning the recombinant proteins at their C-terminus and N-terminus together with the peptide recognition sequences and nucleophilic peptide sequences and then expressing them.

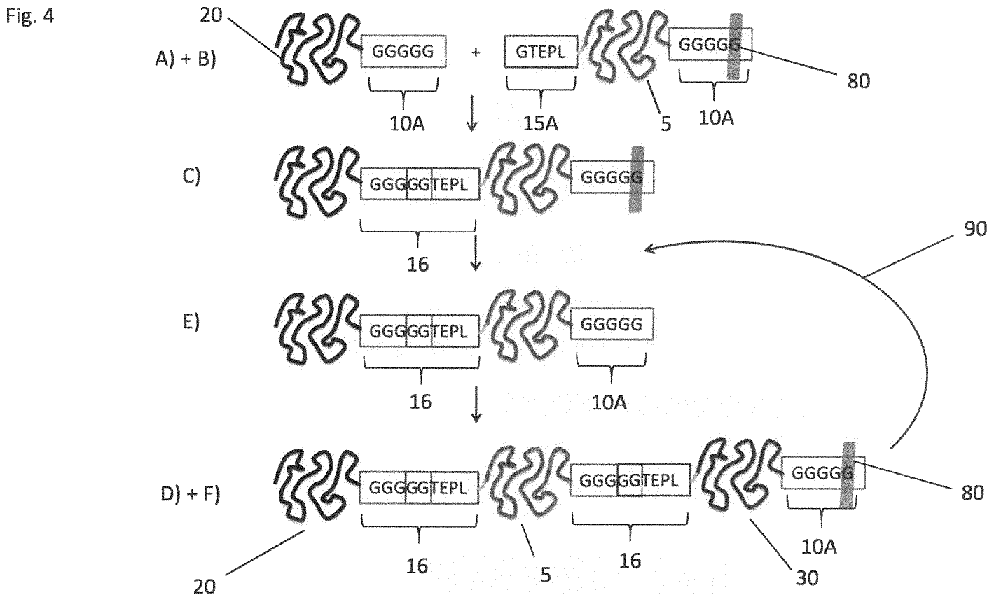

FIG. 4 shows a schematic process of a production method according to the invention for the formation of block copolymers, wherein the peptide recognition sequences and nucleophilic peptide sequences are shown in greater detail. As an example, the recognition sequence and nucleophilic peptide sequence of the Sortase A srtA of Staphylococcus aureus are shown.

In the method steps A) and B) shown at the top of FIG. 4,first block 5 with a peptide recognition sequence 15Aand a nucleophilic peptide sequence 10A with a protecting group80 and a second block 20 with a nucleophilic peptide sequence 10A are provided for the Sortase A.

In method step C), the two blocks are thenlinked together, wherein the C-terminal glycine of the peptide recognition sequence 15A is cleaved off and a thioester-acylintermediate is formed between the sortase and the first block 5 (not shown in the Fig.). In principle, it is also possible to incorporate further amino acids into the peptide recognition sequence at its C-terminus, which are later cleaved off during ligation. By subsequent nucleophilic attack of the nucleophilic sequence 10A on this intermediate product,both blocks 5 and 20 are linked via the then formedpeptide intermediate sequence 16.

In a method step E), the protecting group 80 is then cleaved off, so that the nucleophilic peptide sequence 10A on block5 is now also available for a further coupling reaction.

By means of further ligation reactions, further blocks, for example a third block 30, can then be linked to the already existing linked blocks 20 and 5 in method steps D) and F). In the present case, the linkage between the nucleophilic peptide sequence 10A of block 5 and the peptide recognition sequence of the new block 30 is also performed by Sortase A. The nucleophilic sequence of the third block 30 hasa protecting group 80. This can then be cleaved off againin a further step, designated by the reference number 90, sothat further blocks can then be linked to the growing block copolymer chain.

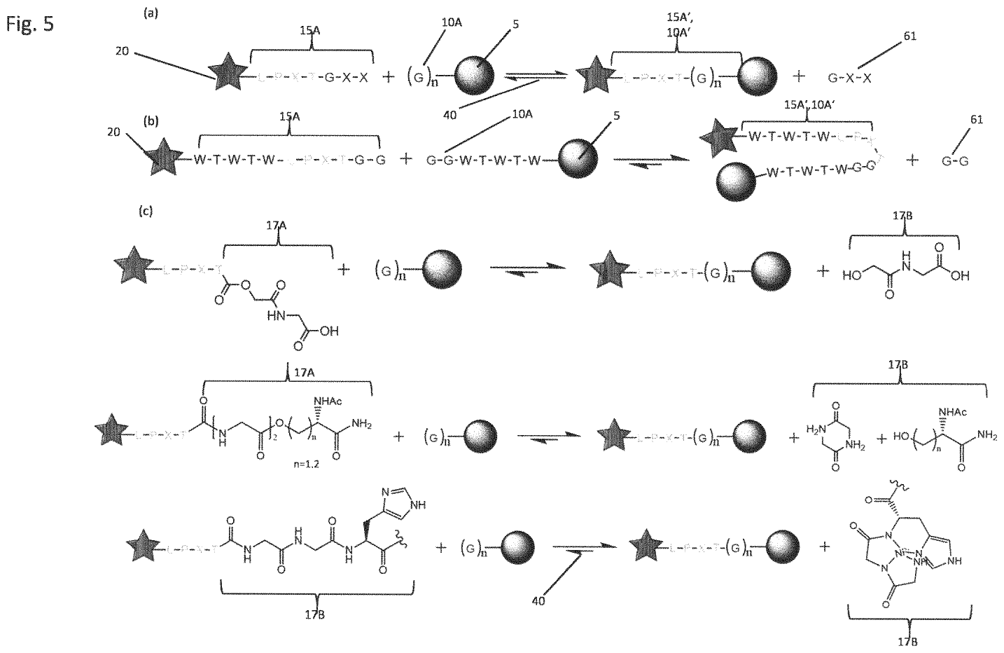

FIG. 5 schematically shows an example of a sortase- catalyzed linkage of two polymer blocks 5 and 20, designated with “(a)”, which have a peptide recognition sequence 15A and a nucleophilic peptide sequence 10A. The equilibrium arrow40 indicates the equilibrium reaction catalyzed by the sortase. When both blocks 5 and 20 are linked, anoligopeptide, 61, is also cleaved off. Since the sortase 40 catalyzes an equilibrium reaction, the block copolymer formed from the polymer blocks 5 and 20 can also be cleaved again as a nucleophile by attack of the cleaved-off oligopeptide6l, whereby the starting compounds arere-formed.

Reference “(b)” is used to designate analternative reaction of a production method according to the invention in which both the peptide recognition sequence 15A and the nucleophilic peptide sequence 10A have a spacer to theblocks 20 and 5 with the peptide sequence -WTWTW-(SEQ ID NO:17). After formation of the reaction product, the peptide intermediate sequence 15A′,10A′ folds due to the spacers to a beta hairpin whichis no longer or only with difficulty accessible to areverse reaction with the sortase.

Further possibilities to shift the equilibrium reactionin the direction of the formation of the product, the linked blocks 5 and 20 are described in FIG. 5 with “(c)”. In each case, chemical groups 17A are coupled to the C-terminal end of the peptide recognition sequence, which, after formation of the product as weak nucleophiles 17B, are not or onlywith difficulty able to cleave the product formed from theblocks 5 and 20 again.

In the following, the production of block copolymers bymeans of methods according to the invention is described.Blocks containing non-peptide polymers, in particularplastics, can be linked as completely artificial blocks with further artificial non-peptide polymers or with artificially produced nanoparticles. In detail, polymer-polymer copolymers of polyethylene glycol (PEG) and poly(N-isopropyl acrylamide)(PNIPAM), nanoparticles surface-modified withPEG or nanoparticle-nanoparticle conjugates areproduced.

### Formation of Nanoparticle-Polymer Block Polymers and Copolymers and Polymer-Polymer Block Copolymers:

Nanoparticles were produced by a sol-gel method with diameters of approximately 200 nm and 60 nm. For this purpose 28 to 30 percent ammonia solution, distilled water and ethanol with molar concentrations of 5 mol/1 for the water, 0.2 mol/1 for the aqueous ammonia solution and 0.2mol/1 for tetraethyl orthosilicate for the nanoparticles with a diameter of 200 nm were produced. For the nanoparticleswith a diameter of 60 nm, 1 mol/1 of water, 0.2 mol/1 of aqueous ammonia solution and 0.2 mol/1 of tetraethyl orthosilicate were used. First a mixture of ethanol, millipore water and ammonia was produced and after 30 minutes equilibration tetraethyl orthosilicate was added inthe indicated amounts. The mixtures were kept at room temperature for 24 hourswith a total volume of 50 ml ethanol.

After formation of the dispersion, 3-(trimethoxysilyl)-propyl methacrylate was added to functionalize the surfacesof the nanoparticles at room temperature, forming a C═C doublebond coating on the surface of the nanoparticles. After 12 hours of stirring, the mixture was refluxed for 1 hour to ensure covalentbonding.

The peptide synthesis of the peptide sequences with the nucleophilic sequence can be performed with a MultiPepRSi synthesizer from INTAVIS Bioanalytical Instruments AG (Cologne) according to a standard Fmoc(Fluorenyl-methoxycarbonyl) protocol. The resin used was either Fmoc-G pre-loaded or unloaded linked polystyrene and the solvent used was dimethylformamide (DMF). The amino acids were activated with HBTU (2-(1H-benzotriazol-1-yl)-1,1,3,3- tetramethyluronium-hexa-fluorophosphate) in DMF/NMM(N-methylmorpholine) and reacted with the peptide 2 timesin 4- fold excess for 90 min each. The Fmoc protecting groupswere cleaved off with piperidine and can be omitted as required after binding the last Fmoc-G derivative.

The peptides were separated from the resin by shaking in 2m1 trifluoroacetic acid (TFA)/triisopropylsilane(TIPS)/water (92.5:5:2.5 V/V) solution. The peptides were then washed by precipitation in 40 ml ice-cold diethyl ether and centrifugation(2×5000 rpm, 4° C.) and isolated. The cleaning was performed with an automated HPLC/ESI MS system. The synthesis of the peptides with thepeptide recognition sequence was carried out analogously, but without a protecting group.

The nanoparticles (NP) with a diameter of 200 nm were functionalized with a peptide of the sequence H-Cys-Ile-Arg- His-Met-Gly-Phe-Pro-Leu-Arg-Glu-Phe-Leu-Pro-Glu-Thr-Gly-OH (peptide 1 for peptide recognition sequence of Sortase A)and the nanoparticles (NP) having a diameter of 60 nm were functionalized with a peptide of the sequence H-Gly-Gly-Gly-Gly-Gly-Phe-Glu-Arg-Leu-Pro-Trp-Phe-Trp-Gly-Met-His-Arg-Ile-Cys-OH (peptide 2 for nucleophilic peptide sequence of Sortase A). For this purpose, the nanoparticles and the 10 peptides were mixed in a molar ratio of 1.1:1 (in relation to the functional groups, in the case of nanoparticles, the number of double bonds on their surface) in 3 mol% of 4,4′- azobis(4-cyanovaleric acid) in water. The mixture was stirred in a nitrogen atmosphere for 24 hours and exposed to UV light 15 (365 nm). The nanoparticle-peptide conjugates formed were then washed with distilled water, centrifuged and ultrasonically cleaned.

Poly(ethylene glycol)methyl ether acrylate (PEGMA)and 20 maleimide terminated poly(N-isopropylacrylamide) (PNIPAM) were coupled with peptides of the sequence H-Gly-Gly-Gly-Gly-Gly-Trp-Phe-Trp-Cys-OH (peptide 3 with the nucleophilic peptide sequence for Sortase A) or with peptides of the sequenceH-Cys-Ile-Arg-His-Phe-Leu-Pro-Glu-Thr-Gly-OH

(peptide 4 with the peptide recognition sequence for Sortase to form the polymer-peptide conjugates. The polymersand peptides were stirred in a molar ratio of 1.1:1 in a pH 7.4 buffer solution under a nitrogen atmosphere at room temperature for 24 hours. Theresulting products were 30 dialyzed twice against Millipore water for 24 hoursusing a dialysis membrane with a MWCO of 2KDa.

To form the nanoparticle-polymer conjugates or polymer- polymer conjugates, 30 μl aqueous solution of the two substrates having an excess of about 50 times of the substrate with the nucleophilic peptide sequence relative to the substrate with the peptide recognition sequence was typically used in a reaction volume of 100 μl.Furthermore, 20 μl Sortase A (7.95 mg/ml), 20 μl 250 mM Tris-HCl (pH 7.5), 750 mM NaCl, 20 μl 25 mM CaCl2, 10 μl Millipore water either at 28° C. (PEG-PNIPAM conjugates) or 37° C. (NP-PEG conjugates, NP-NP conjugates) were mixed in a thermomixer for 24 hours.

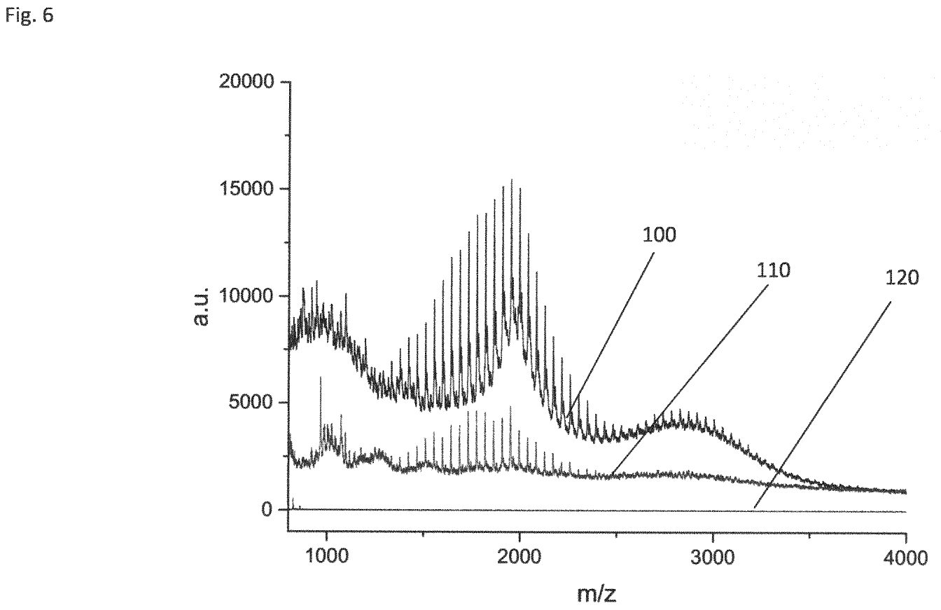

FIG. 6 shows MALDI-ToF mass spectra of a reaction mixture of a linkage of nanoparticles with plastics with Sortase A and negative controls. The curve with thereference number 110 shows only unmodified free polymer fromthe reaction mixture, which cannot be bound to the nanoparticles because it has neither a peptide recognition sequence nor a nucleophilic sequence. In the mass spectrum, a negative control without Sortase A is also inserted, whichshows both unmodified free polymer and thepeptide-polymer conjugate (100). The curve with reference number 120 shows a negative control with nanoparticles and sortase, but without polymer.

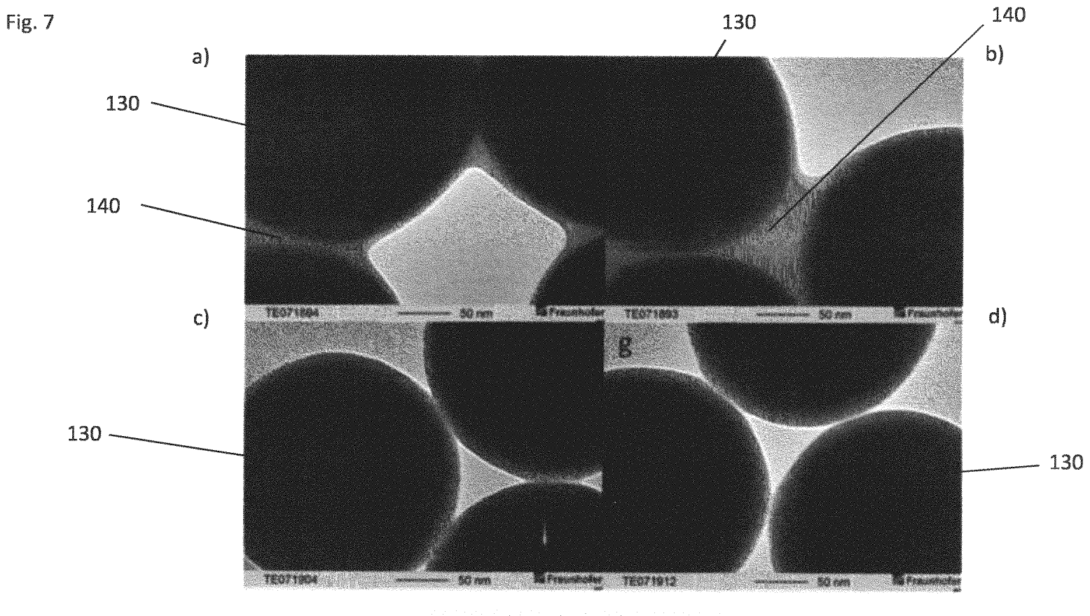

FIGS. 7a to 7d show transmission electron microscope (TEM) images of nanoparticle-polymer conjugates after the Sortase A reaction (FIGS. 7b and 7b), and images of nanoparticles in a negative control without polymer (FIG. 7c) or a negative control without Sortase A (FIG. 7d). The nanoparticles 130 and the polymer coating 140 on the nanoparticles are clearly visible in FIGS. 7a and 7b. In contrast, the negative controls in FIGS. 7c and 7d show no polymer coating 140.

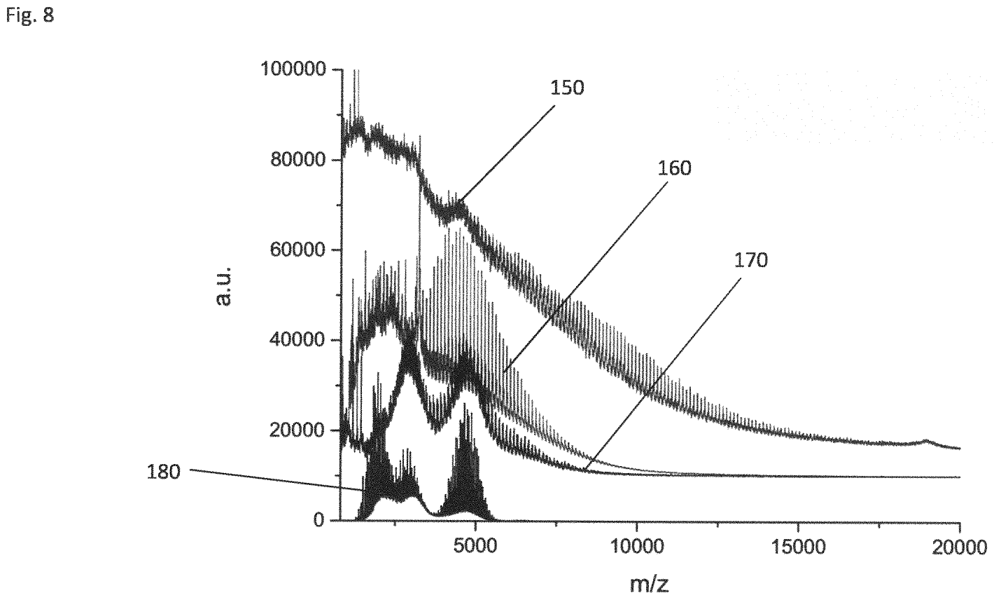

FIG. 8 shows a MALDI-ToF mass spectrum of non-peptide polymers, namely polyethylene glycol PEG linked to peptide 3 (curve with reference number 180), PNIPAM linked to peptide4 (curve with reference number 160), a negative control without Sortase A enzyme (reference number 170), and theproduct of the Sortase A reaction, thePEG-PNIPAM conjugate (reference 150). This mass spectrum therefore clearly shows that a peptide bond is formed between the two polymers by means of Sortase A.

Synthesis of Polymer Blocks with Nucleophilic Peptide Sequences and Peptide Recognition Sequences via RAFT Polymerization:

The synthesis of polymer building blocks with apeptide motif for sortase-mediated ligation is explained in more detail below. The polymerization technique chosen in this case is the “reversible addition fragmentation chaintransfer” (RAFT) polymerization, which allows the synthesis of any polymer components for transpeptidase-mediated ligation, for example by means of a sortase, in which the polymers can be synthesized by RAFT polymerization. This allows both the polymerization of different monomers and the variation ofthe molecular weight of the polymers. For thispolymerization method, a chain transfer agent (CTA) is bound to a peptide terminus and the polymerization is then carried out. The short peptide sequences are also soluble in some organic solvents and have no decisive secondary or tertiary structure, so that the polymerizations can take place in a variety of solvents.

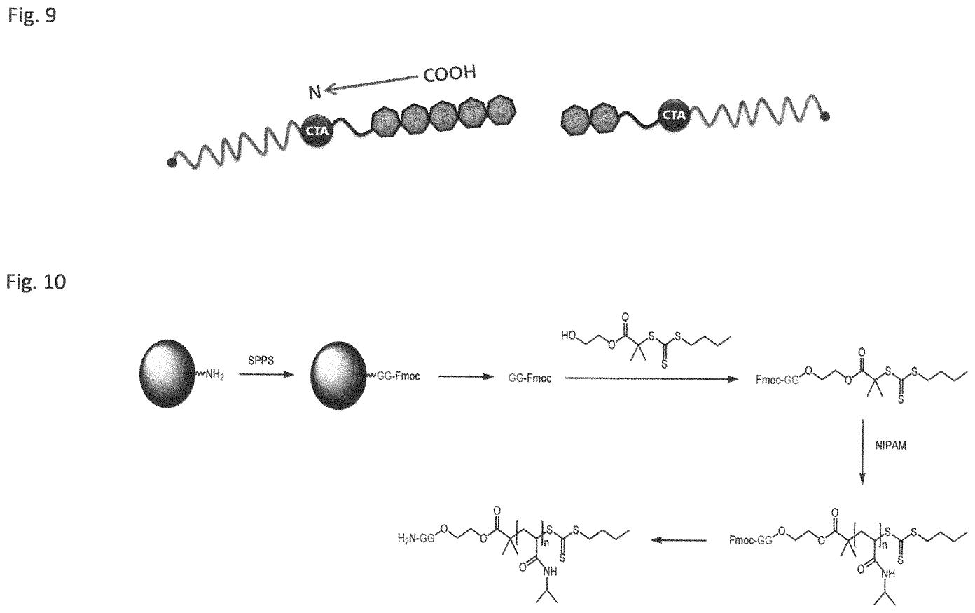

The peptide conjugates are required both withbinding of the polymer blocks to the c- and N-terminus of thepeptides (FIG. 9). For this purpose, a distinction must be made between two synthesis paths. For the nucleophilic peptide sequence, (G)n with n =1-5, the polymer must be bound tothe C-terminus of the peptide and for the peptiderecognition sequence to the N-terminus. The peptide sequencescan be produced with a peptide synthesizer. The solid phase peptide synthesis takes place from C-terminus to N-terminus.

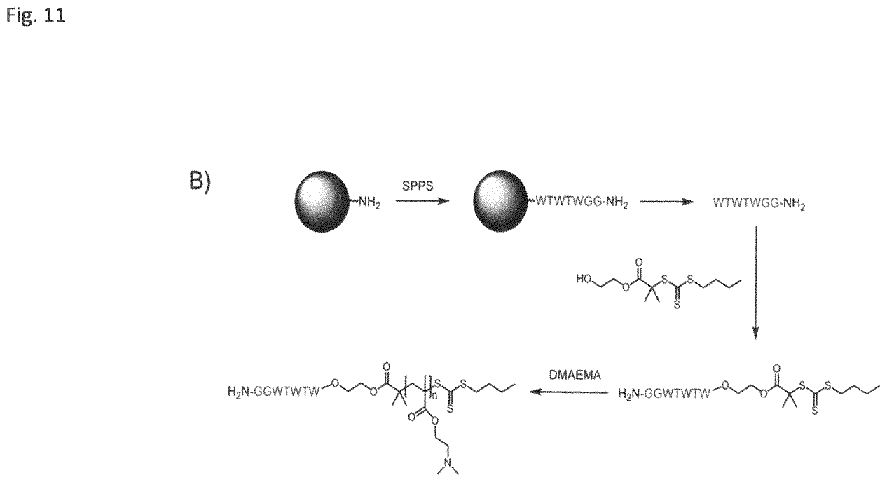

(G)n-CTA can only be produced in two steps. The synthesis path includes the peptide synthesis, peptidecleavage and purification, and binding of RAFT-CTA for subsequent polymerization reaction (FIG. 10 and Fig.11).

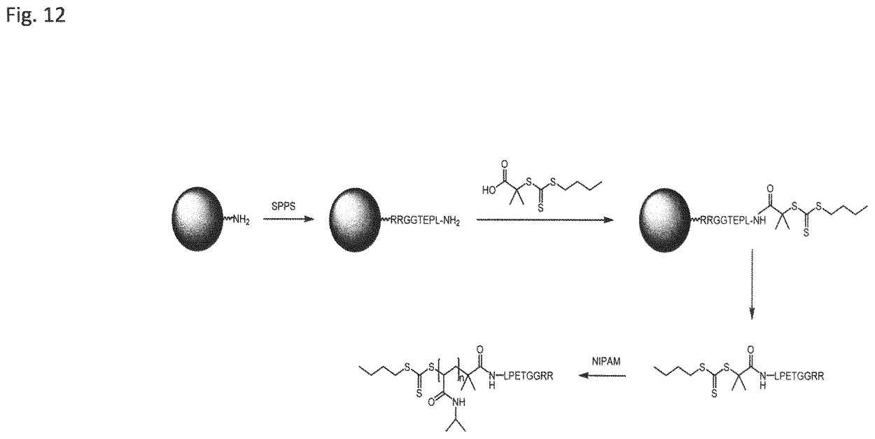

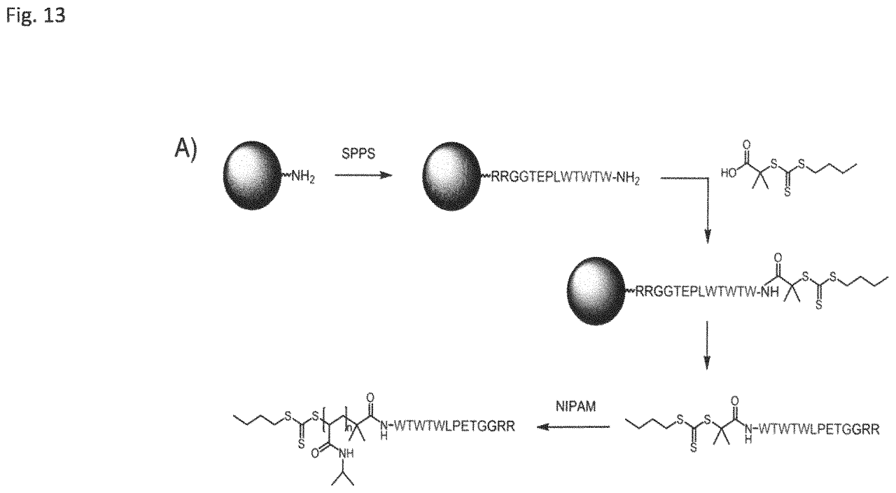

The synthesis can be extended to GGWTWTW-polymer conjugates (FIG. 11) (SEQ ID NO:109). FIGS. 12 and 13 show the synthesisonly with GG and then with GGWTWTW (SEQ ID NO: 109) for the beta hairpin structures.

In the synthesis of polymer-WTWTWLPETGGRR (SEQ ID NO:111) conjugates, a functionality can be bound to the N-terminus directly in the synthesizer as the last step of the peptide synthesis (FIGS. 12 and 13). A suitable RAFT agent will be bound to the SrtA recognition sequence. FIGS. 12 and 13 show the synthesis only with LPETG (SEQ ID NO: 2) and then with WTWTWLPETG for the beta hairpinstructures.

In the following the synthesis of a GG peptide bound to a chain transfer agent (CTA) with an Fmoc protecting group and the subsequent synthesis of a peptide-polymer block is described:

### Synthesis Instructions ForFmoc-GG-CTA:

N-Fmoc-L-glycylglycine (720 mg, 2.04 mmol), 2 hydroxyethyl 2-(((butylthio)carbonothioyl)thio)-2-methylpropanoate (60 mg, 0.20 mmol) and N,N′-dicyclohexylcarbodiimide (DCC) (618mg, 3.0 mmol) were dissolved in 6 mL THF in a flask. The mixture was stirred and cooled with an ice bath for 5 minbefore the addition of 4-(dimethylamino)pyridine (DMAP) (72 mg, 0.6 mmol). The flask remained in the ice bath for 1 hand the reaction mixture was then stirred overnight at RT. The solution was added to approx. 20 mL diethylether, filtered, washed with 5%sodium hydrogen carbonate solution (3 times, 20 mL) and water (2 times, 20 mL). The organic phase was dried over anhydrous magnesium sulfate, filtered and the solvent removed in vacuum. The product was isolated by column chromatography with silica gel and ethyl acetate: pentane (1:1, v/v) as eluent and obtained in 43% yield (56 mg). 1H- NMR (500 MHz, CDCl3, 298 K) o: 7.77 (d, J=7.5 Hz, 2H, Ar- H), 7.60(d, J=7.1 Hz, 2H, Ar-H), 7.41 (m, 2H, Ar-H),7.32 (m, 2H, Ar-H), 4.46 (m, 2H, -CH2-), 4.33 (s, 2H, -CH2-), 4.23 (t, J=6.8 Hz, 1H, -CH-), 4.06 (d, 2H, -CHr), 3.94 (s, 2H, -CHr), 3.26 (t, J=7.4 Hz, 2H, -CHr),1. 69 (s, 6H, -(CH3)2), 1.54 (m, 2H, -CHr), 1.42 (m, 2H, -CHr), 1.25 (s, 2 H , - CHr), 0.92 (t, 3H, -CH3).

### Synthesis Instructions for Fmoc-GG-PNIPAM:

Polymerization of NIPAM from Fmoc-GG-CTA (with a targeted degree of polymerizationof 130): 174.5 mg NIPAM (1.54 mmol), 7.5 mg Fmoc-GG-CTA (0.012 mmol), 5 mL dioxane and 0.6 mg ABCVA (2.14 μmop were placed together with a stir bar in a Schlenk flask. The reaction vessel was then degassedby five freeze-evacuate-thaw cycles. The reaction mixture washeated to 90° C. using an oil bath and stirred in a nitrogen atmosphere for 6 hours. The flask was then kept inliquid nitrogen to stop the reaction. The mixture was transferredto a centrifuge tube and an excess of diethyl ether was addedto precipitate the PNIPAM. The polymer was washed twice with diethyl ether and dried invacuum.

Synthesis Instructions forGG-PNIPAM:

Cleavage of Fmoc: Fmoc-GG-PNIPAM was dissolved in 2 mL piperidine: dichloromethane (1:1, v: v) and stirred for 12 hours. Afterwards diethyl ether was added to precipitate the polymer. The precipitate was washed with diethyl ether and isolated by centrifugation.

The invention is not limited by the description based onthe exemplary embodiments. Rather, the inventionincludes each new feature and each combination of features, which in particular includes each combination of features in the patent claims, even if that feature or combination itselfis not explicitly described in the patent claims orexemplary embodiments.

1. A method for production of block polymers comprising a first block and a second block, comprising method steps of:
A) providing the first block having a nucleophilic peptide sequence for a first transpeptidase enzyme,
B) providing the second block having a peptide recognition sequence for the first transpeptidase enzyme,
C) linking the first block to the second block by means of the first transpeptidase enzyme, wherein the first and second blocks are independently selected from nanoparticles, non-peptide polymers, and recombinant proteins.

2. The method according to claim 1, wherein the second block comprises a first further nucleophilic peptide sequence.

3. The method according to claim 2, wherein the first further nucleophilic peptide sequence is blocked by a protecting group.

4. The method according to claim 3,wherein the first further nucleophilic peptide sequence is for the first transpeptidase enzyme.

5. The method according to claim 2, further comprising, for production of a block polymer having at least three blocks, further method steps of:
D) providing a third block having a peptide recognition sequence for the first transpeptidase enzyme,
E) if present, removing the protecting group of the first further nucleophilic peptide sequence of the second block,
F) linking the third block to the second block by means of the first transpeptidase enzyme.

6. The method according to claim 2, wherein the first further nucleophilic peptide sequence is for a second transpeptidase different from the first transpeptidase.

7. The method according to claim 6, further comprising, for production of a block polymer having at least three blocks, further method steps of:
G) providing a third block having a peptide recognition sequence for the second transpeptidase enzyme,
H) linking the third block to the second block by means of the second transpeptidase enzyme.

8. The method according to claim 5, wherein the third block comprises a nucleophilicsequence for a transpeptidase.

9. The method according to claim 5, further comprising, for the production of block polymers having at least four blocks, after method step F) at least one further block each having a nucleophilic and a peptide recognition sequence for one or more transpeptidase enzymes is provided and bound to the third block, and when the at least one further block is multiple further blocks, the multiple further blocks are linked to one another by means of these one or more transpeptidase enzymes and are bound to the third block.

10. The method according to claim 1, wherein for production of block copolymers, blocks of different structure are used for at least two blocks of the first block and the second block, and for a third block and further blocks when present.

11. The method according to claim 1, wherein synthetic polymers are used for the first block and the second block, and for a third block and further blocks when present.

12. The method according to claim 1, wherein non-peptide biopolymers, independently selected from polyhydroxybutyrates, cellulose, chitin, starch, polylactides and carbohydrates andcombinations thereof, are used for the first block and the second block, and for a third block and further blocks when present.

13. The method according to claim 1, wherein peptide biopolymers, independently selected from spider silk proteins, collagen, keratin and combinations thereof, are used for the first block and the second block, and for a third block and further blocks when present.

14. The method according to claim 13,wherein spider silk proteins are used for the first block and the second block, and for the third block and the further blocks when present.

15. The method according to claim 1, further comprising, for production of branched block polymers, at least one of the first block and the second block, and a third block and further blocks when present, comprises at least three sequences selected from peptide recognition sequences and nucleophilic sequences for a transpeptidase enzyme for binding additional blocks.

16. The method according to claim 1, wherein the peptide recognition sequences are independently selected from the following sequences:
-LPXTG (SEQ ID NO. 1), - LPXTA (SEQ ID NO:10), -NPQTN (SEQ ID NO: 4), -QVPTG (SEQ ID NO: 6), -LAXTG (SEQ ID NO:8), LPXSG (SEQ ID NO: 12), -NHV, -DHV, -(Y)zRH, -(M/L/V)(P/T/A/S)X(A/L/T/S/V/I)G (SEQ ID NO: 94), IPKTG (SEQ ID NO: 88) , APKTG (SEQ ID NO:89), DPKTG (SEQ ID NO: 90), SPKTG (SEQ ID NO:

91. , APATG (SEQ ID NO: 92)
and LPECG (SEQ ID NO: 93), wherein X is any possible amino acid and parameter z is 0 or 1.

17. The method according to claim 1, wherein the nucleophilic sequences are independently selected from -(G)1_s (SEQ ID NOs: 13-14), -(X)₁₋₅- and -(A)15, (SEQ ID NOs: 15-16) wherein X is any amino acid.

18. The method according to claim 1, wherein the transpeptidase enzyme or enzymesare independently selected from: sortases, orfragments thereof, including Sortase A, Sortase B, Sortase C, Sortase D, Sortase E, and butelase, and combinations thereof.

19. A method for the production of block polymers, comprising method steps:
A1) providing a plurality of blocks having a nucleophilic sequence and a peptide recognition sequence for at least one transpeptidase enzyme,
B1) linking the plurality of blocks by the at least one transpeptidase enzyme, wherein the plurality of blocks is independently selected from: nanoparticles, non-peptide polymers, and recombinant proteins.

20. The method according to claim 19, wherein in at least some of the plurality of blocks the nucleophilic sequence is blocked by a protecting group, and in the method step B1), prior to the linking, the protecting groups are removed.

21. The method according to claim 19, wherein the plurality of blocks are linked in the method step B1) step by step, block by block.

22. The method according to claim 19, wherein in the method step B1) the plurality of blocks are linked in a one-pot process.

23. A block polymer having at least a first block 1 and a second block 2 comprising the following structure:
block 1- peptide intermediate sequence 1—block 2 wherein the peptide intermediate sequence 1 bridging block 1 and block 2 contains a sequence or is a sequence selected from the following group:
-(I/L)(P/A)XT(G)₁₋₅-(SEQ ID NOs: 18-22), -(I/L)(P/A)XT(A)₁₋₅-(SEQ ID NOs:23-27), -NP(Q/K)T(G)₁₋₅-(SEQ ID NOs: 28-32), -NP(Q/K)T(A)₁₋₅-(SEQ ID NOs:33-37), -QVPT(G)₁₋₅-(SEQ ID NOs: 38-42), -QVPT(A)1-5-(SEQ ID NOs: 43-47), -LAXT(G)₁₋₅-(SEQ ID NOs: 48-52), -LAXT(A)₁₋₅-(SEQ ID NOs: 56-60), -LPXS(G)₁₋₅-(SEQ ID NOs: 61-65), -LPXS(A)₁₋₅-(SEQ ID NOs: 66-70),
-N(X)₁₋₅-(SEQ ID NOs:71-73), -D(X)₁₋₅-(SEQ ID NOs: 74-76), -LPNT(G)₁₋₅-(SEQ ID NOs: 77-81), -LPNT(A)₁₋₅-(SEQ ID NOs:82-87), IPKT(G)₁₋₅ (SEQ ID NO: 95), IPKT(A)₁₋₅ (SEQ ID NO:96), APKT(G)₁₋₅ (SEQ ID NO: 97), APKT(A)₁₋₅ (SEQ ID NO: 98), DPKT(G)₁₋₅ (SEQ ID NO: 99), DPKT(A)₁₋₅ (SEQ ID NO: 100), SPKT(G)₁₋₅ (SEQ ID NO: 101), SPKT(A)₁₋₅ (SEQ ID NO:

102. , APAT(G)₁₋₅ (SEQ ID NO: 103), APAT(A)₁₋₅ (SEQ ID NO: 104) and LPEC(G) ₁₋₅ (SEQ ID NO: 105), LPEC(A)₁₋₅ (SEQ ID NO: 106), (M/L/V) (P/T/A/S)A(A/L/T/S/V/I)(G)₁₋₅ (SEQ ID NO: 107) and (M/L/V) (P/T/A/S)A(A/L/T/S/V/I)(A)₁₋₅ (SEQ ID NO: 108), wherein X is any possible amino acid,
and wherein block 1 and block 2 are independently selected from: nanoparticles, non-peptide polymers, and recombinant proteins.

24. The block polymer according to claim 23 comprising at least a third block 3: 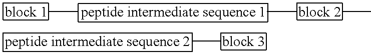
wherein the peptide intermediate sequence 2 independently of the peptide intermediate sequence 1 contains a sequence or is a sequence selected from the following group:
-(I/L)(P/A)XT(G)₁₋₅-(SEQ ID NOs: 18-22), -(I/L)(P/A)XT(A)₁₋₅-(SEQ ID NOs:23-27), -NP(Q/K)T(G)₁₋₅-(SEQ ID NOs: 28-32), -NP(Q/K)T(A)₁₋₅-(SEQ ID NOs:33-37), -QVPT(G)₁₋₅-(SEQ ID NOs: 38-42),-QVPT(A)₁₋₅-(SEQ ID NOs: 43-47),-LAXT(G)₁₋₅-(SEQ ID NOs:
48-52), -LAXT(A)₁₋₅-(SEQ ID NOs: 56-60), -LPXS(G)₁₋₅-(SEQ ID NOs: 61-65), -LPXS(A)₁₋₅-(SEQ ID NOs: 66-70),
-N(X)₁₋₅-(SEQ ID NOs:71-73), -D(X)₁₋₅-(SEQ ID NOs: 74-76), -LPNT(G)₁₋₅-(SEQ ID NOs: 77-81), -LPNT(A)₁₋₅-(SEQ ID NOs:82-87), IPKT(G)₁₋₅ (SEQ ID NO: 95), IPKT(A)₁₋₅ (SEQ ID NO:96), APKT(G)₁₋₅ (SEQ ID NO: 97), APKT(A)₁₋₅ (SEQ ID NO: 98), DPKT(G)₁₋₅ (SEQ ID NO: 99), DPKT(A)₁₋₅ (SEQ ID NO: 100), SPKT(G)₁₋₅ (SEQ ID NO: 101), SPKT(A)₁₋₅ (SEQ ID NO:

102. , APAT(G)₁₋₅ (SEQ ID NO: 103), APAT(A)₁₋₅ (SEQ ID NO: 104) and LPEC(G)₁₋₅ (SEQ ID NO: 105), LPEC(A)₁₋₅ (SEQ ID NO: 106), M/LN)(P/T/A/S)A(A/L/T/SN/1)(G)₁₋₅ (SEQ ID NO: 107) and -(M/LN)(P/T/A/S)A(A/L/T/S/V/1)(A)₁₋₅ (SEQ ID NO: 108), and wherein block 1, block 2 and block 3 are independently selected from:
nanoparticles, non-peptide polymers, and recombinant proteins.

25. The block polymer according to claim 23, having n additional repeat units of a general structure: [-peptideintermediatesequence—block] having the general structure: 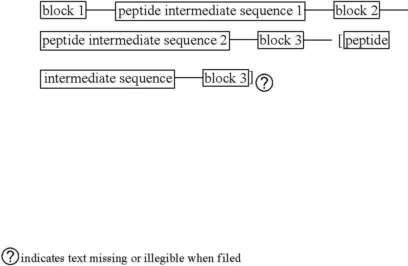
wherein then additional peptide intermediate sequences are independently selected from the corresponding group as claimed in claim 23, and
wherein then additional blocks are independently selected from the corresponding group as claimed in claim 23, and
wherein n is an integer between 1 and 50.

26. The block polymer according to claim 23, wherein the non-peptide polymers are independently selected from polymethyl methacrylates, polyethylene glycols, polyacrylamides and further polymers composed of vinylic monomers.
